(19) United States
(12) Patent Application Publication
(10) Pub. No.: US 2018/0163261 A1
(43) Pub. Date: Jun. 14, 2018

(54) METHODS AND APPARATUSES FOR IMPROVING MUTATION ASSESSMENT ACCURACY

(71) Applicant: ASURAGEN, INC., Austin, TX (US)

(72) Inventors: Robert ZEIGLER, Austin, TX (US); Dennis WYLIE, Austin, TX (US); Brian HAYNES, Austin, TX (US); Gary LATHAM, Austin, TX (US)

(21) Appl. No.: 15/553,125

(22) PCT Filed: Feb. 26, 2016
(86) PCT No.: PCT/US16/19766

§ 371 Date: Aug. 23, 2017

Related U.S. Application Data
(60) Provisional application No. 62/120,923, filed on Feb. 26, 2015.

(51) Int. Cl. C12Q 1/6827

(52) U.S. Cl. CPC C12Q 1/6827

(57) ABSTRACT

Embodiments are provided that relate to methods, systems, kits, computer-readable medium, and apparatuses comprising a computer-based variant calling model that incorporates the viable template count of the aliquot in calling a sequence of a target region based on a set of sequence reads.

### CROSS-REFERENCE TO RELATED APPLICATIONS

This application claims the benefit of priority of U.S. Provisional Patent Application No. 62/120,923, filed Feb. 26, 2015, which is hereby incorporated by reference in its entirety.

### BACKGROUND OF THE INVENTION

### A. Field of the Invention

The present invention relates generally to the field of nucleic acid assays, and more particularly, to the incorporation of a viable template count parameter into a computer-based variant calling model, which may be used in conjunction with assays that involve the chemical and/or physical manipulation of nucleic acid molecules. Embodiments include methods and products involving a variant calling algorithm with viable template count assessment to improve the accuracy of variant calling.

### B. Description of Related Art

Limitations in the availability of many clinical specimens drive the need for low DNA inputs into molecular assays. For example, next-generation sequencing (NGS) is a cutting edge technology that can push the boundaries of input DNA material required for in-depth molecular profiling, particularly in cancer (Beltran, et al., 2013, Menon, et al., Tuononen, et al., 2013, Hadd et al., 2013). With capabilities to accurately detect point mutations, structural variation, copy number changes, methylation status and gene expression, NGS is a multifaceted and versatile tool; however, high sensitivity, high specificity single-nucleotide variant (SNV) calling in NGS of tumor samples is a challenging problem. The input samples are typically heterogeneous, containing mixtures of normal and tumor material, where the tumor material may itself be comprised of a heterogeneous population of cells. Thus it is imperative that any variant detection algorithm achieve high sensitivity with very low variant frequencies to avoid missing real mutations. Variant calling is further challenged by low-quality and low-quantity inputs which elevate background noise to levels on par with biological variants. Thus any method for SNV calling must also achieve high specificity to avoid over-calling samples. A particularly challenging type of input samples include formalin-fixed, paraffin-embedded (FFPE) tumor DNA. FFPE presents a dual challenge for mutation testing, namely requirements for low template input quantities combined with template damage from the fixation and embedding process that resist amplification by PCR. In addition, low quality FFPE DNA can trigger allele dropouts and produce inaccurate results (Didelot et al., 2013, Akbari, et al., 2005).

To start addressing some of the challenges of establishing quality control metrics that can guide reliable sequencing results, entities such as the Next-generation Sequencing Standardization of Clinical Testing (Nex-StoCT) workgroup (coordinated by the Centers for Disease Control), and the College of American Pathologists have proposed criteria for assuring quality NGS data and interpretations. For example, Nex-StoCT recommended a series of post-analytical QC metrics relevant to NGS, including depth and uniformity of coverage, transition/transversion ratio, base call quality score, mapping quality, and others (Gargis et al., 2012).

To date, many methods have been published for variant calling. These generally fall into two classes: tumor-only and matched tumor-normal. Matched tumor-normal algorithms are attractive because they make it possible to discern between biological or “real” mutations that are germline events vs. real mutations which are somatic events. However, in clinical practice, matched samples are more costly to sequence and are often not obtained. Thus, it becomes imperative to have a method which can be run without the corresponding normal sample and still achieve high sensitivity and specificity. Some groups have suggested using simultaneous evaluation of multiple samples from the same tissue, multiple genomic sequences across multiple population members, or genetically related subjects to evaluate the probability of one or more hypotheses being correct (U.S. Publications 2012/0208706, 2014/0057793, and 2014/0058681). Others have suggested using read properties computed for the read of the genetic sequence to evaluate if the reads are unstable or deviate from the typical range of values (EP 2602734A1). Validating NGS output by selectively validating regions of the sample DNA has also been suggested (EP 2602734A1). Several groups have recently described approaches developed specifically for low-level somatic mutations in DNA samples (Hadd et al., 2013, Forshew et al., 2012, Yost et al., 2012), including methods that accommodate sample DNA ‘noise’ such as an elevation in transition mutations (Hadd et al., 2013). However, there remains a need for improving sequencing algorithms and NGS variant calling algorithms.

### SUMMARY OF THE INVENTION

Embodiments include apparatuses, systems, computer readable medium, kits, and methods that overcome the aforementioned limitations and others. The disclosure focuses on the incorporation of the viable template count of a sample in post sequencing analysis to reduce sample input requirements while preserving high sensitivity and positive predictive value (PPV). Additional improvements include targeting either DNA or RNA loci and enabling an operator to go from extracted nucleic acid to sequencing in a short amount of time, including quality control steps. Moreover, integration of the pre-sequencing quality control with the post-sequencing analytics enriches the sequence analysis with sample-specific details that are difficult or impossible to infer from the sequencing data alone, such as the integrity of the nucleic acid or the number of amplifiable copies of nucleic acid input into the library prep.

Some embodiments disclosed herein involve a method comprising quantifying the viable template count in a sample comprising nucleic acid; enriching target regions of the nucleic acid to create a library for sequencing; generating sequence data from the library, wherein the data comprise a plurality of sequence reads; analyzing the sequence data using a computer-based variant calling model that incorporates the viable template count of the sample in calling a sequence of a target region based on a set of sequence reads. It is contemplated that the variant calling model may be implemented by a computing device capable of accessing sequencing data and carrying out the instructions comprised in the variant calling model.

In some embodiments, the variant calling model is configured to call one or more sequence variations in the sample nucleic acid relative to a reference sequence. The sequence variations called by the variant calling model include, but are not limited to, single nucleotide variants, insertions, deletions, multi-nucleotide substitutions, structural variants, genomic copy number alterations, genomic rearrangements, splicing variants, and/or RNA variants. The variants may represent germline mutations, somatic mutations, or both. In some embodiments, the one or more sequence variations are associated with a disease state and/or disease propensity. It is contemplated that methods disclosed herein may be used in the diagnosis and/or prognosis of a variety of diseases or conditions or in ascertaining an individual's propensity for or likelihood of developing a disease or condition. The diseases or conditions may include those that have a genetic component and/or those for which an individual's nucleic acid sequence information would be useful in diagnosing, prognosing, or prescribing a treatment for the disease or condition. It is also contemplated that the methods disclosed herein may be used in predicting an individual's pharmacogenomic response such as resistance, sensitivity, and/or toxicity to a drug. In some embodiments, the variant calling model is configured to identify quantitative target-specific copy number variations.

It is contemplated that in some embodiments disclosed herein, the nucleic acid for which a variant calling model makes sequence and/or variant calls can be derived from a variety of biological and/or synthetic sources. In some embodiments, the nucleic acid comprises DNA, RNA, and/or total nucleic acid from a biological sample. In some embodiments, the nucleic acid comprises genomic DNA. Non-limiting examples of sources from which the nucleic acid can be derived include: formalin fixed paraffin embedded tissue, tissue collected by fine needle aspiration, frozen tissue, serum, plasma, whole blood, circulating tumor cells, tissue collected by laser capture microdissection, core needle biopsy, cerebrospinal fluid, saliva, buccal swab, stool samples, and urine. In some embodiments, the nucleic acid in the sample is heterogeneous. Such heterogeneous nucleic acid may include nucleic acid molecules that have a relatively large amount of sequence in common with other molecules in the sample but vary at some locations. Compositions and samples that comprise heterogeneous nucleic acid can result, for example, from the presence in the sample of different alleles of a gene in a genomic DNA sample; from the nucleic acid in the sample being derived from different sources, such as when some of the nucleic acid is derived from cells in which a somatic mutation has arisen and some is derived from cells in which the same somatic mutation has not arisen; or, in the case of mRNA, from different splicing variants being present in the sample. In some embodiments, the nucleic acid in the sample is from a mixture of cancer cells and non-cancer cells.

In some embodiments, the sample comprising nucleic acid used in generating a library for sequencing has a viable template count below about 10000, 9000, 8000, 7000, 6000, 5000, 4000, 3000, 2000, 1000, 500, 400, 300, 200, 100, or 50. In certain aspects the viable template count is between 10, 20, 30, 40, 50, 100 and 150, 200, 300, 400, 500, 1000, 2000 or more, including all values and ranges there between. In some embodiments, quantifying the viable template count comprises performing a quantitative PCR assay.

Some embodiments disclosed herein involve enriching certain target regions of nucleic acid in a sample to create a library for sequencing. A library is a collection of nucleic acid molecules that comprise the input into a sequencing reaction. The library molecules can serve, for example, as a template for a sequencing reaction that involves replication of at least a portion of the library molecules. A library may be designed to be enriched for certain target regions of, for example, a genome. That is, the library may have more copies of a target region than of a non-target region. In some embodiments, the library may include substantially only target regions, the bulk of the non-target nucleic acid having been removed by a purification process. In some embodiments, enriching target regions of the nucleic acid to create a library comprises performing a PCR reaction using one or more DNA primer pairs capable of annealing and extending over a target region. In some embodiments, the PCR reaction is a multiplex reaction. In some embodiments, enriching target regions of the nucleic acid comprises performing a capture-hybridization procedure.

In some embodiments disclosed herein, generating sequence data from a library comprises obtaining a plurality of sequence reads in parallel. This can be achieved by a number of next generation sequencing platforms. In some embodiments, the sequence data include multiple sequence reads for each portion of the library. In some embodiments, the method further comprises aligning the sequence data to a reference sequence.

Some embodiments disclosed herein involve using a variant calling model that incorporates the viable template count of the sample in calling a sequence of a target region based on a set of sequence reads. A variant calling model can incorporate the viable template count in a variety of different ways that will improve the accuracy and usefulness of the model. In some embodiments, the variant calling model is configured to adjust the probability of a sequence hypothesis being true based on the value of the viable template count. In some embodiments, the variant calling model is configured to downgrade the probability of a sequence hypothesis being true if the variant template count is below a threshold. In some embodiments, the variant calling model is configured to upgrade the probability of a sequence hypothesis being true if the variant template count is above a threshold. In some embodiments, the variant calling model is configured to adjust the weight assigned to a model feature based on the value of the viable template count. In some embodiments, the variant calling model is configured to compare the sequence data to a reference sequence. A reference sequence can include historical or other sequencing information that provides a baseline relative to which variants can be called. In some embodiments, the variant calling model is configured to adjust the prior probability of observing a non-reference base as a function of the viable template count. In some embodiments, the variant calling model is configured to incorporate the viable template count as a feature of the model. That is, the viable template count itself can be a feature of a variant calling model. In some embodiments, the variant calling model is configured to use a different set of model features to identify sequence variants in the sample if the viable template count lies within a predefined interval. In some embodiments, the variant calling model is configured to use an alternative classifier to identify sequence variants in the nucleic acid if the viable template count lies within a predefined interval, e.g., the viable template count is between 10, 20, 30, 40, 50, 100 and 150, 200, 300, 400, 500, 1000, 2000 or more, including all values and ranges there between. Thus, not only can the viable template count itself be a feature of a variant calling model, but it can also influence other features of the model and the way in which the model takes other features into account.

Embodiments described herein take advantage of the inventors' discovery that incorporating viable template count into a variant calling model makes the model more accurate and useful than it would be otherwise. In some embodiments, the variant calling model used in methods described herein has an increased positive predictive value (“PPV”), a decreased incidence of false positives, and/or a decreased incidence of false negatives relative to the same variant calling model that does not incorporate the viable template count. In some embodiments, the variant calling model has a PPV for samples having a viable template count below 200, 100, 75, 50, or 25 and/or above 5, 10, 25, 50, 75 or 100, including all values and ranges there between, that is at least approximately 5, 10, 15, 20, 25, 30, 35, 40, 45, or 50% higher than the same variant calling model that does not incorporate the viable template count. In some embodiments, the variant calling model has a sensitivity for samples having a viable template count below 100 that is no more that 10% less than the same variant calling model that does not incorporate the copy number. In some embodiments, the variant calling model has a PPV above 75% for samples having a viable template count below 100, 200, 300, 400, or 500; or in the range of 10, 20, 30, 40, 50, or 60 to 100, 200, 400, or 500. In some embodiments, the variant calling model has a decreased risk of false positives for samples having a viable template count less than 100, 150, or 200; or in the range of 10, 20, 30, 40, or 50 to 100, 150, 200. In some embodiments, the variant calling model has increased sensitivity for samples having a viable template count above about 1000, 2000, 3000, 4000, or 5000; or in the range of 1000, 2000, 3000, 4000, or 5000 to 6000, 7000, 8000, 9000, or 10000 and does not have a substantial decrease in PPV for those samples relative to the same variant calling model that does not incorporate the viable template count.

In some embodiments, a nucleic acid-containing sample used in the methods disclosed herein comprises DNA derived from a human subject. Nucleic acid is “derived from a human subject” if the nucleic acid was produced in the human subject's body. In some embodiments, a method described above further comprises determining whether the human subject has a disease or a disease propensity based on the analysis of the sequence data. In some embodiments, the disease is cancer. In certain aspects the methods are used to identify a subject with a particular disease or condition, or a subject that may respond in a positive or negative manner to a particular therapy or treatment by assessing the variants in a nucleic acid sample from the subject using the variant calling methods described herein. In some embodiments, the method further comprises selecting a disease treatment based on the analysis of the sequence data. In some embodiments, the disease treatment is administering anti-cancer therapy. Anti-cancer therapy can include, for example, administering a drug, chemotherapy, radiation, and/or surgery. In some embodiments, the method further comprises electing not to administer a disease treatment based on the analysis of the sequence data. In some embodiments, the method further comprises determining whether a disease treatment would be indicated or contraindicated for the human subject based on the analysis of the sequence data.

Also disclosed is a method of improving a computer-implemented variant calling model configured to make sequence calls by analyzing sequence data, the method comprising modifying the model by incorporating into the model's analysis of sequence data a viable template count value for an input sample. In some embodiments, the viable template count value is based on a quantitative PCR assay. In some embodiments, the quantitative PCR assay measures amplification of a DNA fragment that is of a similar size to PCR amplicons in a library from which sequence data analyzed by the model are derived. In some embodiments, incorporating a viable template count into the model's analysis of sequencing data comprises configuring the model to adjust the probability of a sequence hypothesis being true based on the value of the viable template count. In some embodiments, incorporating a viable template count into the model's analysis of sequencing data comprises configuring the model to downgrade probability of a sequence hypothesis being true if the variant template count is below a threshold, e.g., 100, 50, 40, 30, 20, or 10. In some embodiments, incorporating a viable template count into the model's analysis of sequencing data comprises configuring the model to upgrade the probability of a sequence hypothesis being true if the variant template count is above a threshold (e.g., 50, 100, or 200). In some embodiments, incorporating a viable template count into the model's analysis of sequencing data comprises configuring the model to adjust the weight assigned to a model feature based on the value of the viable template count. In some embodiments, incorporating a viable template count into the model's analysis of sequencing data comprises configuring the model to adjust the prior probability of observing a non-reference base as a function of the viable template count. In some embodiments, incorporating a viable template count into the model's analysis of sequencing data comprises configuring the model to incorporate the viable template count as a feature of the model. In some embodiments, incorporating a viable template count into the model's analysis of sequencing data comprises configuring the model to use a different set of model features to identify sequence variants in the sample if the viable template count lies within a predefined interval. In some embodiments, incorporating a viable template count into the model's analysis of sequencing data comprises configuring the model to use an alternative classifier to identify sequence variants if the viable template count lies within a predefined interval. In some embodiments, the modified variant calling model has an increased PPV, a decreased incidence of false positives, and/or a decreased incidence of false negatives relative to the variant calling model before modification. In some embodiments, the modified variant calling model has a PPV for input DNA with a copy number below 100, 75, 50, or 25; or between 5, 10, 15, or 20 and 25, 50, 75 or 100 that is at least approximately 5, 10, 15, 20, 25, 30, 35, 40, 45, or 50% higher than the variant calling model before modification. In some embodiments, the modified variant calling model has a sensitivity for input samples having a viable template count less than 100 that is no more that 10% less than the sensitivity of the variant calling model before modification. In some embodiments, the modified variant calling model has a PPV above 75% for input aliquots having a viable template count below 100, 200, 300, 400, or 500; or between 5, 15, 25, 50, or 75 and 100, 200, 300, 400, or 500. In some embodiments, the modified variant calling model has a decreased risk of false positives for input aliquots having a viable template count less than 100, 150, or 200 relative to the model before modification. In some embodiments, the method further comprises training the model using a panel of known variants and sequencing data derived from input samples with varying viable template count values, including samples with fewer than about 100 functional DNA copies and samples with more than about 500 functional DNA copies.

Also disclosed is a non-transitory machine-readable storage medium comprising instructions that, when executed by a computing device, cause the computing device to perform at least the following: access sequence data associated with a library of nucleic acid molecules, wherein the library is generated from a nucleic acid input sample; and analyze the sequence data to identify sequence variants by taking into account a viable template count associated with the input sample. Accessing sequence data can include, for example, obtaining sequence data and/or receiving sequence data. In some embodiments, the library comprises nucleic acid molecules enriched from the nucleic acid input sample by PCR and/or capture hybridization. In some embodiments, the enriched nucleic acid molecules are associated with a disease state, a disease propensity, and/or a pharmacogenomic response to drug treatment. In some embodiments, the viable template count has been calculated by a quantitative PCR assay. In some embodiments, the nucleic acid input sample is derived from a biological sample selected from one or more of the following: formalin fixed paraffin embedded tissue, tissue collected by fine needle aspiration, frozen tissue, serum, plasma, whole blood, circulating tumor cells, tissue collected by laser capture microdissection, core needle biopsy, cerebrospinal fluid, saliva, buccal swab, stool samples, and urine. In some embodiments, the input nucleic acid comprises DNA, RNA, and/or total nucleic acid from a biological sample. In some embodiments, the input nucleic acid comprises genomic DNA. In some embodiments, taking into account a viable template count associated with the input sample comprises adjusting the probability of a sequence hypothesis being true based on the value of the viable template count. In some embodiments, taking into account a viable template count associated with the input sample comprises downgrading the probability of a sequence hypothesis being true if the variant template count is below a threshold. In some embodiments, taking into account a viable template count associated with the input sample comprises upgrading the probability of a sequence hypothesis being true if the variant template count is above a threshold. In certain aspects a threshold can be a predetermined number or a calculated number. In some embodiments, taking into account a viable template count associated with the input sample comprises adjusting the weight assigned to a feature of a variant calling model based on the value of the viable template count. In some embodiments, taking into account a viable template count associated with the input sample comprises adjusting the prior probability of observing a non-reference base as a function of the viable template count. In some embodiments, taking into account a viable template count associated with the input sample comprises incorporating the viable template count as a feature of the model. In some embodiments, taking into account a viable template count associated with the input sample comprises using a different set of model features to identify sequence variants in the sample if the viable template count lies within a predefined interval. In some embodiments, taking into account a viable template count associated with the input sample comprises using an alternative classifier to identify sequence variants if the viable template count lies within a predefined interval.

Also disclosed is a kit for determining a nucleic acid sequence comprising: (a) a quantitative PCR reagent set capable of being used to determine the viable template count of nucleic acid in a sample; (b) a multiplexed PCR reagent set capable of being used to amplify multiple target regions in the sample and generating a library of nucleic acid molecules for sequencing; (c) a tagging PCR reagent set capable of being used to append sequences to the nucleic molecules in the library; (d) a set of reagents capable of being used to purify and/or normalize the nucleic acid molecules in the library for further amplification prior to sequencing; (e) a non-transitory machine-readable storage medium comprising instructions that, when executed by a computing device, cause the computing device to identify sequence variants by performing at least the following: (i) access or receive sequence data associated with the library of nucleic acid molecules; and (ii) analyze the sequence data to identify sequence variants by taking into account the viable template count associated with the sample. In some embodiments, the quantitative PCR reagent set comprises a master mix capable of being used to make a buffer suitable for quantitative PCR. In some embodiments, the quantitative PCR reagent set comprises primers for amplifying a region or segment of a nucleic acid in the sample. In some embodiments, the multiplexed PCR reagent set comprises primers configured to amplify at least 5, 10, 15, 20, 25, 30, 35, 40, 45, or 50 genomic regions associated with a disease state or disease propensity. In some embodiments, the genomic regions cover at least 50, 100, 200, 300, 400, 500, 600, 700, or 800 loci associated with a disease state or disease propensity. In some embodiments, the disease is cancer. In some embodiments, taking into account a viable template count associated with the sample comprises adjusting the probability of a sequence hypothesis being true based on the value of the viable template count. In some embodiments, taking into account a viable template count associated with the sample comprises downgrading the probability of a sequence hypothesis being true if the variant template count is below a threshold. In some embodiments, taking into account a viable template count associated with the sample comprises upgrading the probability of a sequence hypothesis being true if the variant template count is above a threshold. In some embodiments, taking into account a viable template count associated with the sample comprises adjusting the weight assigned to a feature of a variant calling model based on the value of the viable template count. In some embodiments, taking into account a viable template count associated with the sample comprises adjusting the prior probability of observing a non-reference base as a function of the viable template count. In some embodiments, taking into account a viable template count associated with the sample comprises incorporating the viable template count as a feature of the model. In some embodiments, taking into account a viable template count associated with the sample comprises using a different set of model features to identify sequence variants in the sample if the viable template count lies within a predefined interval. In some embodiments, a viable template count associated with the sample comprises using an alternative classifier to identify sequence variants if the viable template count lies within a predefined interval.

Also disclosed is a method of identifying variants in a genomic DNA sample comprising: (a) performing a quantitative PCR assay to determine the viable template concentration in a sample comprising nucleic acid; (b) using the viable template concentration to calculate the viable template count in an aliquot of the sample; (c) performing a PCR reaction to create a library enriched for a nucleic acid segment of interest using the aliquot as a template; (d) generating sequence data from the library; and (e) analyzing the sequence data using a computer-based variant calling model that incorporates the viable template count to identify sequence variants in the genomic DNA, wherein incorporating the viable template count comprises configuring the model to do one or more of the following: adjust the probability of a sequence hypothesis being true based on the value of the viable template count; downgrade the probability of a sequence hypothesis being true if the variant template count is below a threshold; upgrade the probability of a sequence hypothesis being true if the variant template count is above a threshold; adjust the weight assigned to a model feature based on the value of the viable template count; adjust the prior probability of observing a non-reference base as a function of the viable template count; incorporate the viable template count as a feature of the model; identify sequence variants in the sample if the viable template count lies within a predefined interval; and/or use an alternative classifier to identify sequence variants in the nucleic acid if the viable template count lies within a predefined interval.

Also disclosed is a method of improving the quality of variant calling of a nucleic acid sample comprising: (i) determining the amount of functional copies in a sample to be sequenced and (ii) determining the amount of sample to be used in sequencing based on the amount of functional copies in the sample. In some embodiments, the functional copies are RNA functional copies. In some embodiments, the determined amount of sample to be used in sequencing comprises at least 100, 200, 300, or 400 functional copies.

In some embodiments, generating sequence data can include obtaining multiple sequence reads in parallel. This can be achieved by, for example, employing next-generation sequencing (NGS) platforms including but not limited to MiSeq, HiSeq, or NextSeq instruments from Illumina, PGM, or Proton instruments from ThermoFisher, and other platforms provided by Roche/Pacific Biosciences, Complete Genomics, Oxford Nanopore, BioRad/GnuBio, Genia, Stratos, Noblegen, Lasergen, and Nabsys.

In some embodiments, the sample comprises RNA and the method involves identifying variants in the RNA in the sample. Such embodiments may include a reverse transcription step before the quantitative PCR step, the step performing PCR to create a library, or both.

In some embodiments described herein, a variant calling model is configured to adjust the probability of a variant hypotheses based on the viable template count. The viable template count may be used as a model feature for evaluating variant hypotheses. Additionally or alternatively, viable template count may be used to adjust the weight or score of another model feature used in evaluating variant hypotheses.

Embodiments also include, but are not limited to, methods, kits, apparatuses, systems, and computer-readable medium for improving the accuracy and/or sensitivity of an assay that identifies genetic variants from a patient, diagnosing a patient with a disease or condition based on identifying one or more genetic variants, diagnosing a patient based on sequencing a plurality of markers, identifying genetic variants in a sample with a low abundance of high quality genetic material, reducing false positive determinations of genetic variants, reducing false negative determinations of genetic variants, using an algorithm that improves variant calling, for determining whether one or more sequences are variants with higher accuracy, using a variant calling model to improve diagnosis or determining the sequence of a potential variant in a biological sample. In various embodiments, a gene sequencing machine is used to identify genetic variants and the sequencing output is evaluated using a trained algorithm that refines the output to take into account whether a sufficient number of good nucleic acid templates were available in the sample that was sequenced. In certain embodiments, systems include the computer hardware to run an algorithm that improves variant calling. Any of these embodiments can be employed with the steps and/or components described in this disclosure.

In certain embodiments, there is a method of diagnosing a patient based on determining whether the patient has genetic variants in a nucleic acid sample obtained from the patient comprising: assaying at least a portion of the nucleic acid sample to determine the number of nucleic acid templates usable in a sequencing reaction involving amplified nucleic acid molecules; amplifying nucleic acid molecules in the sample; sequencing the amplified nucleic acid molecules at one or more regions that includes a potential variant associated with a disease or condition; and using an algorithm to evaluate the data from the sequences amplified nucleic acid molecules.

If a patient is identified as having one or more genetic sequences that indicates a particular treatment regimen, in certain embodiments the patient is treated for a disease or condition associated with the one or more genetic sequences.

It is contemplated that any embodiment discussed in this specification can be implemented with respect to any method, system, kit, computer-readable medium, or apparatus of the invention, and vice versa. Furthermore, apparatuses of the invention can be used to achieve methods of the invention.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting” or “reducing” or any variation of these terms includes any measurable decrease or complete inhibition or reduction to achieve a desired result. The terms “promote” or “increase” or any variation of these terms includes any measurable increase or production of a nucleic acid, protein, or molecule to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The apparatuses and methods for their use can “comprise,” “consist essentially of,” or “consist of” any of the components or steps disclosed throughout the specification.

A “variant” is a form or version of something that differs in some respect from other forms of the same thing or from a standard. When used in reference to a nucleic acid sequence, a “variant” is a nucleic acid that differs in some respect from other forms of the same nucleic acid or from a standard nucleic acid. Non-limiting examples are single nucleotide polymorphisms (SNPs); single nucleotide variants (SNVs); complex base changes, such as multi-nucleotide substitutions; structural variants, genomic copy number alterations and rearrangements, quantitative copy number estimates, and/or combinations thereof. The standard or other form of the same nucleic acid from which the variant differs can be, but are not limited to, a biological nucleic acid, a non-biological nucleic acid, a synthetic nucleic acid, a plant nucleic acid, an animal nucleic acid, a fungi nucleic acid, a prokaryote nucleic acid, a human nucleic acid, a normal tissue nucleic acid, a cancer tissue nucleic acid, a diseased tissue nucleic acid, a prior nucleic acid, a nucleic acid from a genetically related organism or family member, a nucleic acid representing a general or specific nucleic acid found in a population, an artificial nucleic acid, a nucleic acid from a standard, a nucleic acid from another sample in the library, a nucleic acid from the same sample, and/or combinations thereof.

A “variant calling model” or “variant caller” is a set of instructions by which a computer analyzes nucleic acid sequencing data to call a sequence and/or variant in a target nucleic acid molecule (i.e., to indicate a sequence or indicate whether a sequence at a particular position in a target nucleic acid molecule differs or does not differ relative to a reference sequence). In some embodiments, a variant calling model (1) assesses the probability or likelihood that nucleic acid molecules in a sample have sequence variations (i.e., deviations from a reference sequence) and (2) provides information and/or generates a report regarding one or more variants that are likely to be present or absent in a sample and the likely frequency of such variations, if any, in the sample. In some embodiments, a variant calling model indicates the certainty or probability of error of a sequence or variant call, including, in some embodiments, the certainty or probability of error of an indication of no variant at a location.

A first DNA molecule is of a similar size to a second DNA molecule if the first molecule is between about 85 to 115% of the size of the second DNA molecule.

“Viable template” is a nucleic acid that is PCR-amplifiable, amplifiable by any enzymatic process, and/or manipulatable by any protein or protein moiety and is from a sample containing nucleic acids to be assayed by one or more chemical or physical tests.

“Viable template concentration” is the number of viable templates per volumetric unit. In some embodiments, it may be determined using quantitative PCR systems such as QuantideX® qPCR DNA QC Assay. In some embodiments, it may be determined using any other method that reveals a viable template count, including but not limited to real-time PCR, digital PCR, or isothermal amplification methods.

“Viable template count” is the absolute number of viable templates in an aliquot comprising sample nucleic acid. One way that the viable template count for an aliquot can be calculated is by multiplying the viable template concentration of a sample by the volume of an aliquot taken from the sample. The viable template count can also be calculated by any other way that reveals the quantity of viable templates in a composition comprising nucleic acids. In some embodiments, a variant calling model takes the viable template count into consideration in making sequence calls and/or identifying sequence variants.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—The general structure and elements of one embodiment of a contemplated method or kit are shown in the workflow.

FIGS. 2A and B—(A) Components of an embodiment of a contemplated method or kit integrates elements of a PCR-based enrichment workflow with sample quantification and bioinformatics. (B) QuantideX® Pan Cancer DNA panel.

FIGS. 3A and B—(A) Overview of QuantideX® DNA QC methodology. (B) Overview of the entire integrated workflow for RNA and DNA targets, including QuantideX® QC reagents, NGS reagents, other workflow components and the QuantideX®-enabled variant caller. In one embodiment, the QuantideX® NGS system is a streamlined workflow from QC to informatics that enables simultaneous quantification of DNA point mutations, indels, structural variants, RNA expression and gene fusions from a total nucleic acid (TNA) isolated from low-input, low-quality samples. As a non-limiting example, targeted NGS QC can be performed with a novel qPCR assay that quantifies functional DNA and RNA from the total nucleic acid isolated from a sample. PCR-based target enrichment can be conducted using QuantideX® targeted NGS reagents and sequenced on a MiSeq® (Illumina). Library sequences can be analyzed using QuantideX® NGS Reporter, a bioinformatic analysis suite that directly incorporates pre-analytical QC information to improve the accuracy of variant calling, fusion detection and RNA quantification.

FIG. 4—An embodiment of a contemplated method or kit that enables the quantification and enrichment of cancer-related variants of several genes from DNA purified from human tissue or cell-lines. The kit or method supports multiplex next-generation sequencing analysis with a sequencing instrument (Illumina MiSeq instrument demonstrated here). The kit or method includes components for determining QFI Assay Score and Inhibition and Profile software that analyzes sequence files such as FASTQs for the identification of base substitution mutations and small insertions/deletions using a locally integrated bioinformatic pipeline and companion data visualization tools.

FIG. 5—Application of a kit to determine QFI Assay Score and Inhibition Profile to a set of clinical nucleic acids isolations.

FIGS. 6A and B—(A) An example of 2 steps of PCR contemplated in a method and/or kit embodiment: i) gene-specific amplification with a common sequence concatenated to each primer; ii) second PCR appending instrument-specific adaptors and index codes are added to the PCR product. Products from individual samples are pooled then clustered onto the flow cell. After imaging, the index codes are used assign individual sequencing reads to their respective libraries. (B) An example of Dual Index codes (with ILMN adaptors, specific codes, and CS1/CS2 regions) is shown.

FIG. 7—Mastermix Setup: Primer mix (3545-1)-92 primer pairs, 2× PCR mastermix (3469-1) (the same as QuantideX® NGS core reagents), sample at fixed volume of 4 μL; and “Mastermix-free” setup for tagging PCR—oligos as premixture, 2× mastermix (3469-1), and aliquot of gene-specific products.

FIGS. 8A and B—Yield by amplicon, overall coverage and variability between operators highlights performance for the panel using (A) operators 1, 2, and 3 (3.9, 5.3, 6.5% respectively) and (B) paraffin-embedded samples.

FIG. 9—QuantideX® DNA QC reveals elevated false positive mutation calls with limited viable template molecules (QuantideX® Cp#) when applying a variant caller that lacks viable template information.

FIGS. 10A and B—Limited functional copies greatly increases the risk of false positives (right panes) and limits sensitivity (left panes). QuantideX®-enabled caller shows consistent performance across the entire range of functional copy inputs. Asuragen variant caller compared to caller lacking consideration of input copy number reveals a suppression of false positive calls at low functional template copies while retaining high sensitivity to the known positive BRAF V600E (A) and KRAS G12V (B). These samples were not used in training the model.

FIG. 11—Outline of model-building inputs and strategy.

FIG. 12—Performance was evaluated on putative germline and putative somatic variants. Shown is the distribution of percent variants in each group, illustrating that the putative germline variants follow an expected biomodal distribution whereas putative somatic variants are smeared across the entire range with a heavy bias toward low % variant (<25%).

FIG. 13—Sensitivity by allele frequency of various current-generation variant callers, as assessed in http://genomemedicine.com/content/5/10/91/.

FIG. 14—QuantideX®-enabled caller improves PPV between 1% and 100% variant and provides as equivalent or better sensitivity across the same range relative to baseline.

FIG. 15—QuantideX®-enabled caller is sensitive across the entire range of inputs. QuantideX®-enabled calling particularly benefits low-input samples, increasing PPV by 50% relative to the baseline model below 100 copies. Depicted is the performance on putative somatic variants.

FIG. 16—Table of performance on putative germline variants. Baseline model and QuantideX®-enabled models yield equivalent results on this data set.

FIG. 17—In a cohort of over 600 FFPE samples, more than 27% would contain <100 functional copies of DNA using a 10 ng input. The QuantideX® variant caller substantially reduces the risk of false positives in this set relative to baseline and other extant variant callers.

FIG. 18—QuantideX® caller shows extremely high analytical sensitivity, correctly calling as few as 1.7 mutant copies.

FIG. 19—QuantideX® QC reveals the relationship between the % of usable sequencing reads (y-axis) and the functional copies input into the sequencing reaction (x-axis) for 51 FFPE samples of varying quality sequenced with a panel targeting the ERBB2 gene.

FIG. 20—Comparison of copy number variation detection using QuantideX® caller Next Generation Sequencing (NGS CNV) and droplet digital PCR (BioRad, Sep25).

FIG. 21—Standard deviation of within-sample relative amplification efficiencies. As the DNA quality score (QFI) decreases, the relative efficiency differences are exacerbated, leading to elevated deviation from expected baselines.

FIG. 22—Percent functional DNA for any size range (Brisco, et al., 2010) estimates by NGS-based approach compared to qPCR-based method.

FIG. 23—Lower quality samples (graded by the RNA functional copy assay) can be rescued by increasing library mass input.

FIG. 24—RNA Functional copies predicts targeted sequencing data quality for two independent targeted RNA-Seq panels: 40 target mRNA expression panel (left) and 50 target gene fusion panel (right). Libraries prepared with less than 100 viable RNA template molecules show diminished mapping rates to the intended targets and elevated rates of primer dimer formation for both panels.

FIG. 25—RNA functional copies correlate with the reads on target produced by NGS. Three FNAs titrated from 100 ng to 0.01 ng of intact TNA input reveals a stronger correlation between functional RNA template copies and post-sequencing on target mapping rates than the mass inputs and on target mapping rates.

### DESCRIPTION OF ILLUSTRATIVE EMBODIMENTS

As noted above, one of the unique aspects of the present invention is the incorporation of the viable template count of a sample in the post sequencing analysis of sequencing results. This allows for the benefits of reduced sample input requirements while preserving high sensitivity and positive predictive value (PPV), targets both DNA and RNA loci, and enables an operator to go from extracted nucleic acid to sequencing in a short amount of time, including quality control steps. Moreover, integration of the pre-sequencing quality control with the post-sequencing analytics enriches the sequence analysis with sample-specific details that are difficult or impossible to infer from the sequencing data alone, such as the integrity of the nucleic acid or the number of amplifiable copies of nucleic acid input into the library prep.

Determining the percentage or quantity of functional copy numbers or viable template count of nucleic acids in a sample can be used to determine the amount of sample needed to meet the minimum nucleic acids requirement to perform molecular assays (Sah, et al., 2013, WO Publication 2013/159145). To date, several methods for determining the percentage or amount of viable template count of nucleic acids or the frequency of lesions have been published (Sah, et al., Brisco, et al., 2010, Brisco, et al., 2011, U.S. Publication 2012/0322058, WO Publication 2013/159145). For example, it has recently been described that the results of a PCR quantification assay, termed quantitative functional index-PCR or QFI-PCR, can be used to calculate the minimum amount of sample input for molecular assays, such as targeted PCR enrichment, by measuring the number and percentage of DNA templates that are competent for PCR amplification (Sah, et al., 2013). This insight can reduce the risk of false positives and false negatives in variant calling using both laboratory-developed and commercially available procedures for enrichment and subsequent NGS. As a result, the integration of a pre-analytical step based on QFI-PCR offers a much improved approach to ensure accuracy in NGS data interpretations, not only for the evaluation of FFPE DNA prior to NGS, but also for other assays that rely on PCR amplification. Thus, rigorous and quantitative characterization of DNA-poor samples is essential to ensure that results are generated from sufficient copies of functional DNA templates, interpreted with consideration of DNA quality, and can support reliable mutation calls. The consequences of a misguided diagnostic decision based on sequencing results from inadequate amplification of DNA template are serious and could lead to inappropriate patient treatment by failing to identify an actionable mutation or prescribing the wrong treatment based on a false positive result. Such errors may also undermine retrospective biomarker association studies relevant to cancer drug development. However, even the use of QFI-PCR as previously described to determine the appropriate amount of sample DNA needed in PCR based molecular assays does not address all of the challenges in NGS sequence calling of low quality samples.

The following subsections describe non-limiting aspects of the present invention in further detail.

### A. Nucleic Acid Sample

It is contemplated that embodiments described herein can include all types of nucleic acids, including, but not limited to, DNA, RNA, single stranded nucleic acids, double stranded nucleic acids, heterogeneous nucleic acids, homogenous nucleic acids, nucleic acids from normal cells, nucleic acids from cancer cells, nucleic acids from mixtures of normal cells and cancer cells, and/or combinations thereof. Non-limiting examples of sources of nucleic acids include biological sources, non-biological sources, synthetic sources, clinical or non-clinical sources, plasma/serum, fresh tissue, frozen tissue, circulating tumor cells, laser capture micro-dissection (LCM) tissue biopsies, core needle biopsies, fine needle aspiration (FNA) tissue, whole blood, cerebrospinal fluid (CSF), saliva, buccal swab, stool samples, urine, tumors, formalin fixed paraffin embedded tissue (FFPE), and/or combinations thereof. In some aspects the nucleic acid sample may be contained in an aliquot or extraction of a sample that contains nucleic acid.

### B. Determination of Viable Template Count

It is contemplated that embodiments can include all types of methods and apparatuses for determining viable template count.

Non-limiting examples of embodiments for determining viable template count include QFI-PCR, quantitative PCR, real-time PCR, digital PCR, other PCR-based methods that reveals the amplifiable copy number, and non-PCR methods which include, but are not limited to, isothermal amplification, rolling circle amplification, or similar methods, and/or combinations thereof. Additional non-limiting examples include the methods and apparatuses described in U.S. Publication 2014/0051595, Sah, et al., 2013, Brisco, et al., 2010, Brisco, et al., 2011, U.S. Publication 2012/0322058, and WO Publication 2013/159145.

### C. Creation of a Library for Sequencing

It is contemplated that the methods and apparatuses of the present invention can include all types of methods and apparatuses for creation of a library for sequencing. Non limiting examples include enrichment of target regions by any means, PCR-based methods, multiplex PCR based-methods, methods based on capture-hybridization, and/or combinations thereof. It is further contemplated that the library may contain: one or more subgenomic regions of interest; one or more amplified regions of interest; and/or one or more regions of interest associated with any disease, condition, state, pharmacogenomic response (e.g., resistance, sensitivity and/or toxicity), propensity for such, and/or combinations thereof.

### D. Generation of Sequencing Data

It is contemplated that the methods and apparatuses of the present invention can include all types of methods and apparatuses for the generation of sequencing data. Non limiting examples include PCR and non PCR based methods, a MiSeq instrument, a HiSeq instrument, a NextSeq instrument, a PGM instrument, a Proton instrument, a Roche/PacBio platform, an Oxford Nanopore platform, a Complete Genomics platform, a Genia platform, a Stratos platform, a BioRad/GnuBio platform, a Nabsys platform, etc. It is further contemplated that the sequencing data may include one or more sequence reads for each portion of the library and/or no reads for one or more portion of the library. It is also contemplated that the sequencing platform, instrument, or machine may be configured to sequence a single or multiple library segments in series or in parallel.

### E. Variant Calling Model

A variant calling model can be configured with a variety of instructions for determining whether the sequencing data indicate the likely existence of a variant in the sample. As an example, a sequencing read aligned against a reference sequence may indicate that a single nucleotide variant (SNV) exists at a given location in the input DNA. This results in a “variant hypothesis” that the SNV exists at that location. To assess the probability that the input DNA actually does have an SNV at that location (i.e., that the variant hypothesis is true), the variant calling model may be configured to take into account various aspects of the sequencing data as model features, covariates, and/or classifiers for making that assessment. One such criterion may be the proportion of sequencing reads that also indicate the same SNV. The model may instruct the computer that if the proportion is low, the probability of an SNV actually existing in the sample should be downgraded. As another example, the model may be configured to take into account whether the sequencing reads from the complementary strand show the same SNV and adjust the probability of the SNV existing in the input DNA accordingly. A variant calling model can include any number of model features, covariates, and/or classifiers for assessing the probability of a variant. The final list of likely variants and their frequencies is the product of applying all of the model's instructions to all of the variant hypotheses derived from the raw sequencing data.

It is contemplated that the methods and apparatuses of the present invention can include one or more of all types of variant calling models. Non limiting examples of models may include linear models, Linear Discriminant Analysis (LDA), Diagonal Linear Discriminant Analysis (DLDA), Random Forests, Support Vector Machines (SVMs), Logistic regression, Poisson regression, Bayesian networks and other graphical models, Naïve-Bayes, decision trees, boosted trees, k-means clustering and neural networks, Hidden Markov Model (HMMs), and/or combinations thereof. Specific, non-limiting examples of variant calling models include:

SuraScore—a poisson-based model which computes by poisson test the probability of the variant given the underlying quality scores, for bases with quality scores >q15. Spurious variants which arise from low-quality sequencing are down weighted in this scheme and are likely to be classified as negative whereas variants from high-quality sequencing data can be called with high sensitivity and good specificity. This model is good for high-sensitivity detection of low-frequency mutants.

SuraScoreBB—a beta-binomial based genotyping model. This model is good for accurate and sensitive detection of germline SNPs and uses prior probability distribution information derived from historical sequencing data.

It is contemplated that the variant calling model may incorporate the viable template count in any way. Non limiting examples of the means of incorporating viable template count in the variant calling model may include the following means: the model downgrades, upgrades, includes, does not include, or modifies the probability of one or more variants existing in the sample based on the viable template count; the model downgrades, upgrades, includes, does not include, or modifies the weight or use of one or more model features, covariates, and/or classifiers; and/or the model downgrades, upgrades, includes, does not include, or modifies one or more sequence reads used in calling the sequence. Further specific non limiting means of incorporating viable template count in the variant calling model may include the following means:

(1) Direct inclusion of the number of viable template count and/or “QFI” (DNA quality score) which may include, but is not limited to: (A) FunctionalCopiesSample—the number of functional copies reported directly by the viable template count assay; (B) FunctionalCopiesPanel—the number of viable template count of the sample adjusted for the median amplicon size of the sequencing panel using a model which predicts this information from the QFI, the median amplicon size of the panel, and the FunctionalCopiesSample; and (C) FunctionalCopiesAmplicon—the number of functional copies of the sample, adjusted on a per-position basis based on the length of amplicon(s) covering the position, which may utilize a model which predicts functional copies based on QFI and the FunctionalCopiesSample.

(2) Modifications of other scoring metrics in a copy-dependent manner. This class of features may be, but is not limited to being, based on the knowledge that the scoring metrics assume statistical independence between sequencing reads, but this assumption breaks down when insufficient material is put into the initial reaction for library generation. In that case, there is high inter-dependence between the reads. These features are computed in general as:

Copy Adjusted score=Score/max((Coverage/FunctionalCopiesSample), 1);

wherein the FunctionalCopiesSample may be substituted with FunctionalCopiesPanel and FunctionalCopiesAmplicon to create metrics adjusted for the amplicon sizes in the panel or for individual amplicon sizes, respectively.

It is contemplated that the variant calling model may use one or more viable template count thresholds or viable template count range thresholds. Non limiting examples of the viable template count threshold include percentages of total nucleic acid content or copies or number of viable template counts such as: 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, 99%, etc. of total nucleic acid, or any percentage or range derivable therein; or 0, 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, etc., viable template counts or any number or range derivable therein and/or combinations thereof.

It is further contemplated that the variant calling model may be trained. The variant calling model may be trained on any set of data derived from any input nucleic acid. It is contemplated that variants and sequencing data derived from the input nucleic acid may or may not have: uniform, varying, or combinations of copy numbers; uniform, varying, or combinations of viable template count; and/or uniform, varying, or combinations of any other factor considered by the variant calling model.

It is contemplated that all or a portion of the variant calling model may or may not be stored on one or more machine-readable storage medium. It is further contemplated that the one or more machine-readable storage medium may or may not be executed by a local processor, remote processor, through an internet interface, and/or any combination thereof.

### F. Model Features, Covariates, and Classifiers

It is contemplated that the methods and apparatuses of the present invention can include all types of model features, covariates, and/or classifiers. Non limiting examples of model features and covariates may include one or more of: scoring metrics, percent variant, quality-scores, depth of coverage, beta genotyping prior derived from historical data, functional copy input, viable template count, the percentage of guanine (G) and/or cytosine (C) in a defined window up or downstream of the base of interest, the longest homopolymer observed in a defined window up or downstream of the base of interest, a measure of how strong the association is between observing the mutant and the proximity to the end of the read, a measure of how strong the association is between the position within a read a base is at and the likelihood of observing a mutation at the base, the format of the functional copy or viable template assay used, input type into the functional copy or viable template assay used (TNA or DNA), the 95th percentile of percent variant across all hypotheses, coverage of the base at issue relative to the median sample coverage, number of times the base at issue was sequenced, the base identity one base-pair removed in the 3′ direction from the position under consideration, the percent of the ten bases in the 3′ direction from the position under consideration that are guanine (G) and/or cytosine (C), the longest homo-polymer stretch of the ten bases in the 3′ direction from the position under consideration, the percent of the fifteen bases in the 3′ direction from the position under consideration that are guanine (G) and/or cytosine (C), the longest homo-polymer stretch of the fifteen bases in the 3′ direction from the position under consideration, the base identity two base-pairs in the 3′ direction from the position under consideration, the percent of the twenty bases in the 3′ direction from the position under consideration that are guanine (G) and/or cytosine (C), longest homo-polymer stretch of the twenty bases in the 3′ direction from the position under consideration, the base identity three base-pair in the 3′ direction from the position under consideration, the percent of the five bases in the 3′ direction from the position under consideration that are guanine (G) and/or cytosine (C), the longest homo-polymer stretch of the five bases in the 3′ direction from the position under consideration, the number of variants occurring within three positions from the edge of a read, the total number of bases occurring within three position form the edge of a read, the hypothesis-specific 95th percentile of the percent variant, the hypothesis (A>C, G>T, etc.), the global population minor allele frequency of the variant, the median QScore at the position, the trimean of the qscores at that position (average of the 25^{th} percentile, 50th percentile, and 75 percentile of the qscores), the total number of mate pairs covering the position, the base identity one base-pair in the 5′ direction from the position under consideration, the percent of the ten bases in the 5′ direction from the position under consideration that are guanine (G) and/or cytosine (C), the longest homo-polymer stretch of the ten bases in the 5′ direction from the position under consideration, the percent of the fifteen bases in the 5′ direction from the position under consideration that are guanine (G) and/or cytosine (C), the longest homo-polymer stretch of the fifteen bases in the 5′ direction from the position under consideration, the base identity two base-pair in the 5′ direction from the position under consideration, the percent of the twenty bases in the 5′ direction from the position under consideration that are guanine (G) and/or cytosine (C), the longest homo-polymer stretch of the twenty bases in the 5′ direction from the position under consideration, the base identity three base-pair in the 5′ direction from the position under consideration, the percent of the five bases in the 5′ direction from the position under consideration that are guanine (G) and/or cytosine (C), the longest homo-polymer stretch of the five bases in the 5′ direction from the position under consideration, and/or combinations thereof

In one embodiment, all of the model features, covariates, and/or classifiers disclosed in the paragraph above are include in the variant calling model. In a preferred embodiment, all of the model features, covariates, and/or classifiers disclosed in the paragraph above are included in the SuraScore and/or SuraScoreBB variant calling model and the model uses the Copy Adjusted score to adjust the score of one or more model features, covariates, and/or classifiers. Variations of the embodiments are also contemplated.

### G. Sequence Variants

It is contemplated that embodiments can include, predict, call, etc. any sequence variant. Non-limiting examples of sequence variants may include: single nucleotide polymorphisms (SNPs); single nucleotide variants (SNVs); complex base changes, such as multi-nucleotide substitutions; structural variants, genomic copy number alterations and rearrangements, quantitative copy number estimates, and/or combinations thereof. It is also contemplated that the sequence variant of the present invention can be associated with any disease, condition, state, pharmacogenomic response (e.g., resistance, sensitivity and/or toxicity), propensity for such, and/or combinations thereof. Non limiting examples may include cancer, diabetes, obesity, infection, autoimmune diseases, aging, renal diseases, metabolic syndrome, neuropathologies, cerebrovascular disease, Alzheimer's, cardiovascular diseases, stroke, sensitivity to drugs, sensitivity to compounds, sensitivity to complexes, toxicity of drugs, toxicity of compounds, toxicity of complexes, resistance to drugs, resistance to compounds, resistance to complexes, and/or combinations thereof.

It is contemplated that multiple variants may be assayed in parallel or in sequence. In certain embodiments, the number of loci or variants that are assayed may be at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000 loci or variants, or any range derivable therein.

### H. Aligning the Sequence

It is contemplated that embodiments of the present invention can include aligning the sequence data to one or more reference sequence(s). Non-limiting examples of reference sequences include: a biological sequence, a non-biological sequence, a synthetic sequence, a plant sequence, an animal sequence, a fungi sequence, a prokaryote sequence, a human sequence, a normal tissue sequence, a cancer tissue sequence, a diseased tissue sequence, a prior sequence, a sequence from a genetically related organism or family member, a sequence based on general or specific genetics of a population, an artificial sequence, a sequence from a standard, a sequence from another sample in the library, a sequence from the same sample, and/or combinations thereof.

### I. Methods

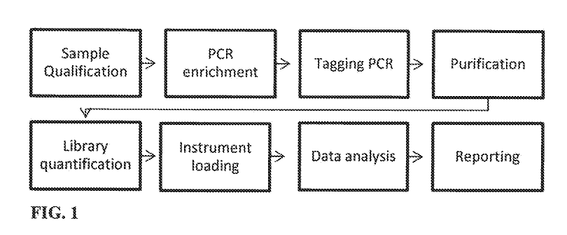

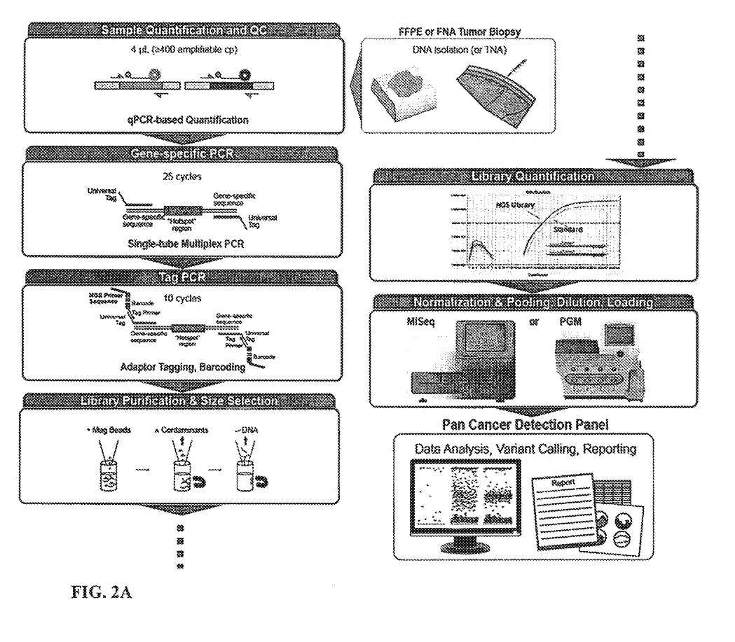

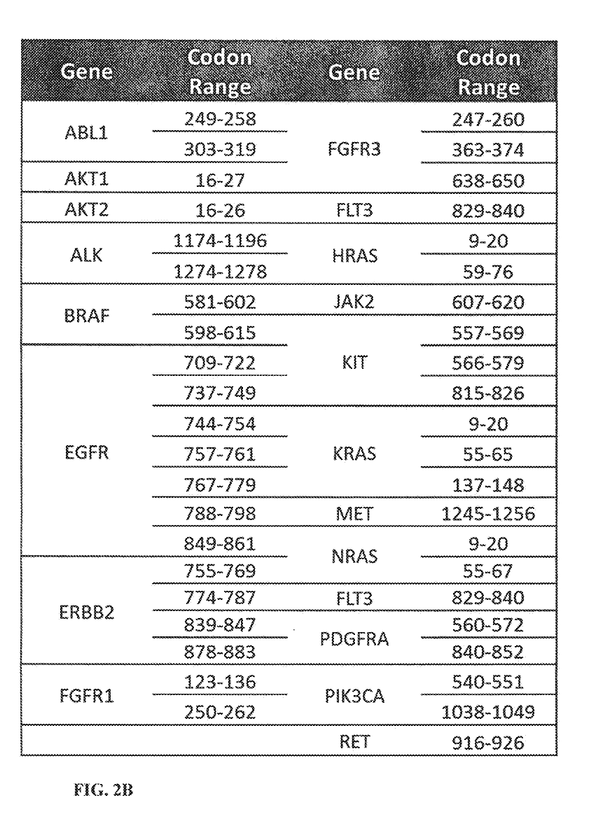

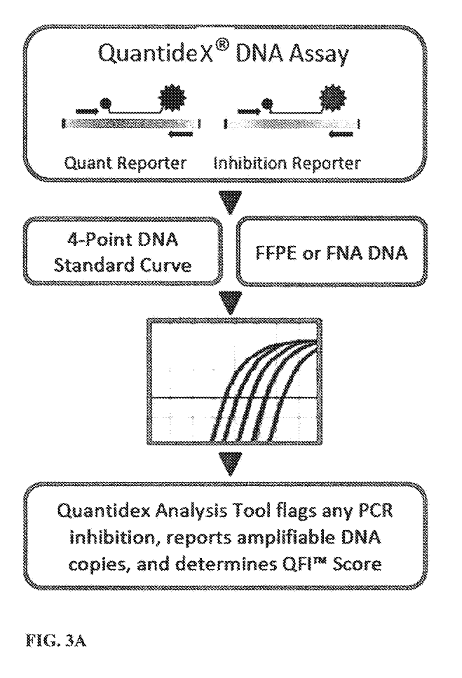

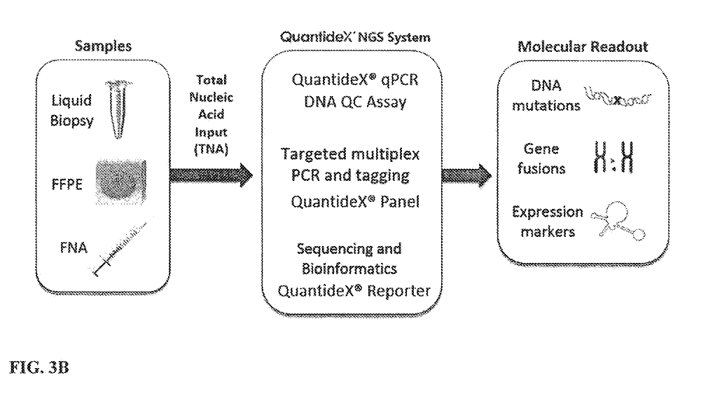

It is contemplated that embodiments of the present invention can include methods and processes. Non-limiting examples of methods include methods for training a variant calling model, methods for incorporating a viable template count into a variant calling model as a model feature, methods for integrating elements of a PCR-based enrichment workflow with sample qualification and bioinformatics. Non-limiting examples of methods of integrating elements of a PCR-based enrichment workflow with sample qualification and bioinformatics include: methods that comprise sample qualification, PCR enrichment, tagging PCR, purification, library quantification, instrument loading, data analysis, and reporting (FIG. 1); methods that comprise a quantification and/or inhibitor assay, such as QuantideX® QC Assay; gene-specific PCR; Tag PCR; purification and size selection; library quantification; normalization and pooling, dilution, and loading; sequencing, such as through the use of MiSeq; and data analysis, variant calling, and reporting, such as through the use of QuantideX® Reporter Bioinformatics (FIGS. 2A and B and FIGS. 3A and B).

### J. Kits

Kits are also contemplated as being used in certain aspects of the present invention. For instance, apparatuses of the present invention can be included in a kit. A kit can include one or more containers. Containers can include a bottle, a metal tube, a laminate tube, a plastic tube, a dispenser, a pressurized container, a barrier container, a package, a compartment, or other types of containers such as injection or blow-molded plastic containers into which the apparatuses or desired bottles, dispensers, or packages are retained. The kit and/or containers can include indicia on its surface. The indicia, for example, can be a word, a phrase, an abbreviation, a picture, or a symbol.

A kit may also include: one or more quantitative PCR reagents; one or more multiplexed PCR reagents; one or more tagging PCR reagents; one or more reagents for purifying and/or normalizing nucleic acids from a sample or the amplified targets; one or more machine-readable storage medium comprising instructions which, when executed by a processor, cause the processor to perform a method for identifying sequence variants from the sequencing data files; one or more instructions providing access to one or more local or remote machine-readable storage medium comprising instructions which, when executed by a processor, cause the processor to perform a method for identifying sequence variants from the sequencing data files; one or more primers, one or more probes, one or more standards, one or more positive and/or negative controls, one or more synthetic batch controls; one or more buffers; one or more diluent; and/or one or more polymerases or other nucleic-acid modifying enzymes.

A kit may also include instructions for employing the kit components, the use of any other product included in the kit, or the use of other products not included in the kit, such as, but not limited to, software or a web based application. Instructions can include an explanation of how to apply, assemble, use, and maintain the products and/or components.

In one instance, a kit may provide components or instructions for integrating elements of a PCR-based enrichment workflow with sample qualification and bioinformatics. In another instance, a kit may follow the following workflow: sample qualification, PCR enrichment, tagging PCR, purification, library quantification, instrument loading, data analysis, and reporting (FIG. 1). In yet another instance, a kit may include components directed to a quantification and/or inhibitor assay such as the QuantideX® DNA QC assay; gene-specific PCR; Tag PCR; purification and size selection; library quantification; normalization and pooling, dilution, and loading; sequencing, such as through the use of MiSeq; and data analysis, variant calling, and reporting, such as through the use of QuantideX® Reporter Bioinformatics (FIGS. 2A and B and FIGS. 3A and B). In one aspect, a kit may enable the quantification and enrichment of cancer-related variants in multiple genes from nucleic acid purified from human tissue or cell-lines. In another aspect, a kit contains or supports one or more of the following: supports multiplex next-generation sequencing analysis with a specific instrument, such as an Illumina MiSeq instrument; includes software that analyzes sequencing data files, such as MiSeq data files, for the identification of base substitution mutations and small insertions/deletions; uses a locally integrated bioinformatic pipeline; and/or uses companion data visualization tools.

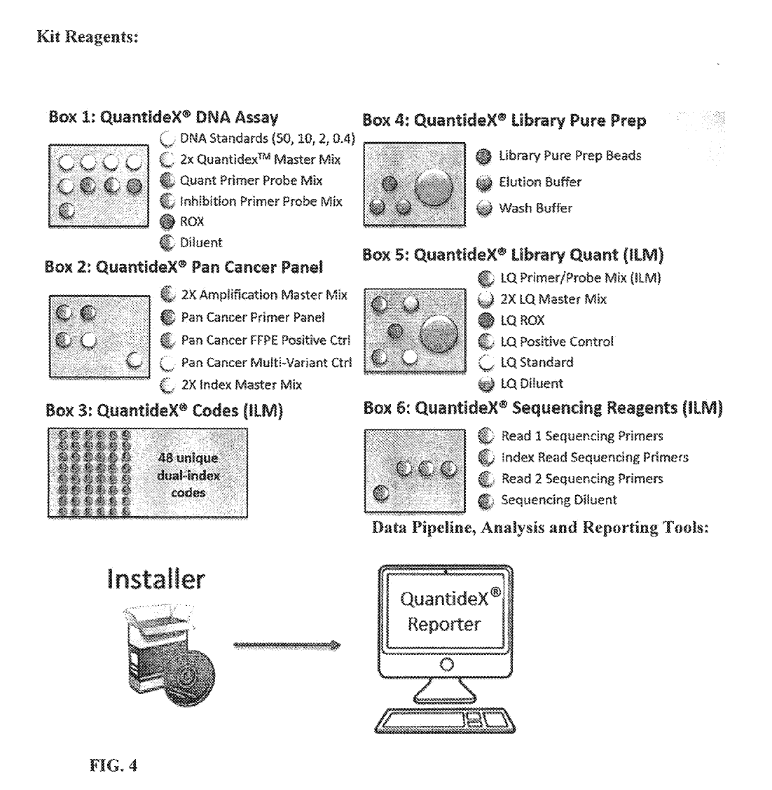

In another aspect, a kit may include one or more of a QuantideX® DNA Assay Kit comprising as an example, primers, probes, ROX, and standards; core reagents such as QuantideX® Pan Cancer primers, a FFPE positive control, a synthetic batch control, Taq, buffer mastermix, diluent; a QuantideX® Bead Purification comprising as an example, QuantideX® beads, elution buffer, wash buffer; a QuantideX® (MiSeq) component comprising as an example, mastermix, ROX, diluent, primers/probes, standards, positive controls, and a calibration means; a MiSeq Index Codes primer mix; a Tagging Reagents and Custom MiSeq primers component comprising as an example, mastermix, diluent, and custom sequencing primers (FIG. 4). In yet another aspect, a kit may comprise or further comprise an installer, and a web or on-site deployed data analysis package for installation as a local application (FIG. 4).

In another instance, a kit may include components to determine viable template count and/or an inhibition profile. In a particular embodiment, such component is a QuantideX® NGS kit. A QuantideX® NGS kit may contain one or more of the following reagents: 2× mastermix with reagents combined in minimum vial set for simple set up and workflow, pre-diluted standards for ease of use and reproducibility, and/or ROX passive dye for instrument compatibility (FIG. 4). In another instance the components to determine viable template count and/or an inhibition profile determines a QFI Assay Score and Inhibition (Cq) (FIG. 5).

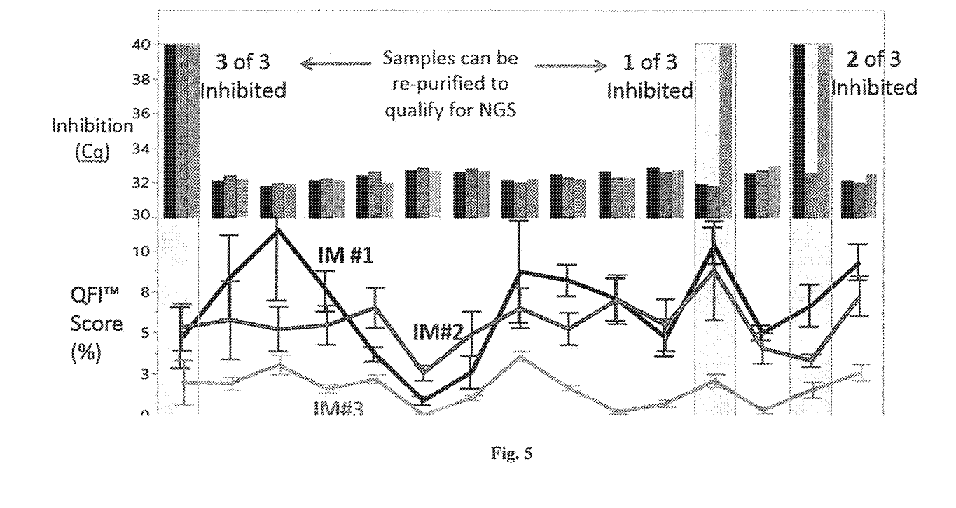

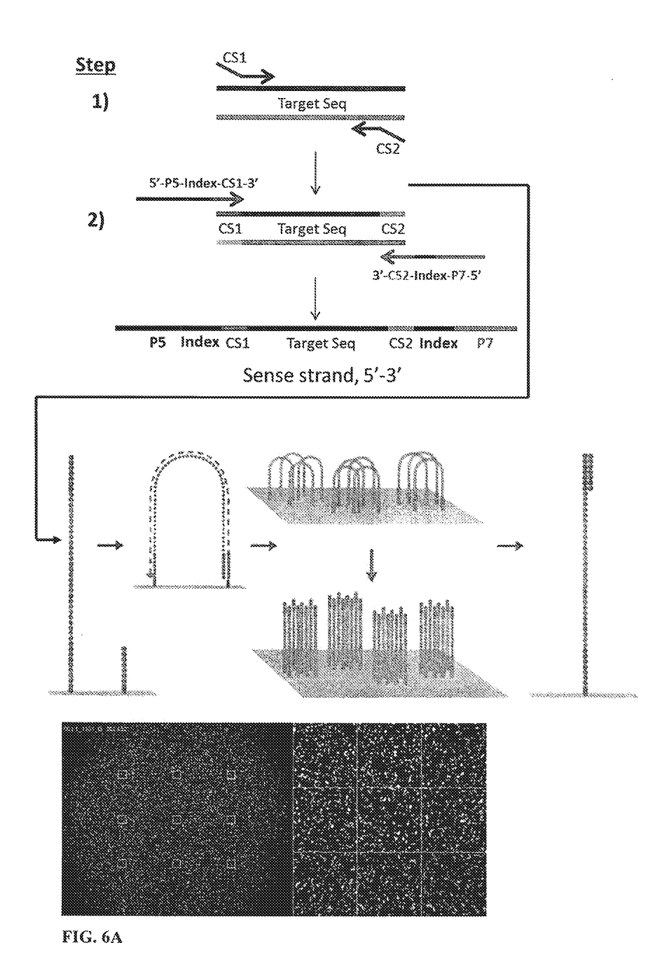

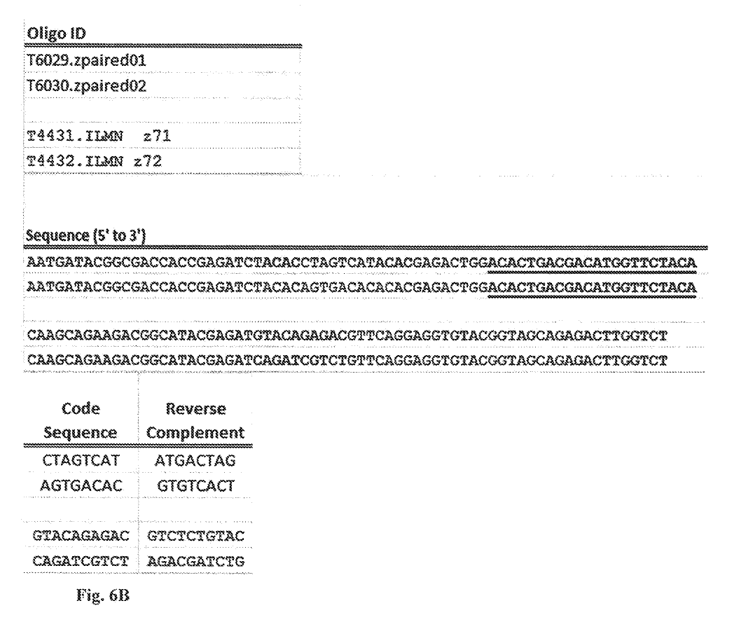

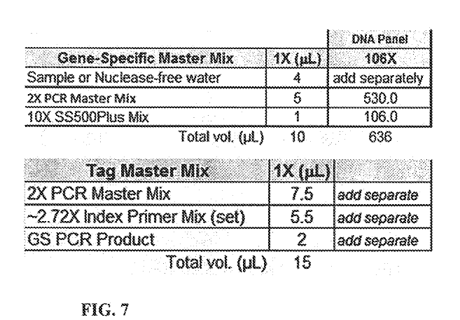

In one aspect, a kit may include a gene specific and tagging PCR. The kit may use a work flow that uses 2 steps of PCR for gene specific and tagging PCR. In another aspect, the 2 steps of PCR may be: (i) gene-specific amplification with a common sequence concatenated to each primer; and (ii) second PCR appending instrument-specific adaptors and index codes are added to the PCR product. In yet another aspect, a kit may further comprise wherein products from individual samples are pooled then clustered onto one or more flow cell(s) and after imaging, index codes are used to deconvolute the identity of each amplicon for each sample (FIGS. 6A and B). In one instance the gene specific and tagging PCR component of a kit includes at least one gene-specific mastermix and a tag mastermix. In another instance the at least one gene-specific mastermix and a tag mastermix comprise the following: Mastermix Setup—primer mix (3545-1) of 92 primer pairs, 2× PCR mastermix (3469-1) same as QuantideX® NGS reagents, sample at fixed volume of 4 μL; and/or “Mastermix-free” setup for tagging PCR—oligos as premixture, 2× mastermix (3469-1) and aliquot of gene-specific products (FIG. 7).

In another aspect, a kit may include target panel and/or positive controls. In one instance, the kit includes a residual clinical FFPE-sourced DNA control. In another instance the process control is formulated from several synthetic DNAs admixed with genomic DNA and representing several different variants. In yet another instance, the kit controls represent cancer-related variants. In one instance the kit controls are formulated form a BRAF V600E positive and “wild-type” tumor.

In yet another aspect, a kit may include a library purification, quantification, and loading component. In one instance, the library purification removes free PCR primers and buffer components and/or reduces non-specific primer dimer products from the multiplex PCR. In another instance, a library quantification is used as an internal quality control check prior to sample loading and/or to normalize the yields between sample libraries prior to pooling. In yet another instance, library purification is performed by bead purification. A non-limiting example of bead purification includes magnetic bead-based purification. In one instance the library quantification method is a calibration-curve free qPCR method. A non-limiting example of a quantification method includes competitive PCR with spiked standard used for concentration determination which uses delta Ct to determine the concentration of each library. In another instance, a loading component is premixed with sequencing primers to specified concentration and supplied with the kit. In yet another instance, for the loading component, a user pools samples, denatures with PhiX, dilutes and loads to cassette. In one instance for a loading component, a user supplies dual-index code list and links QuantideX® results to FASTQ files for analysis.

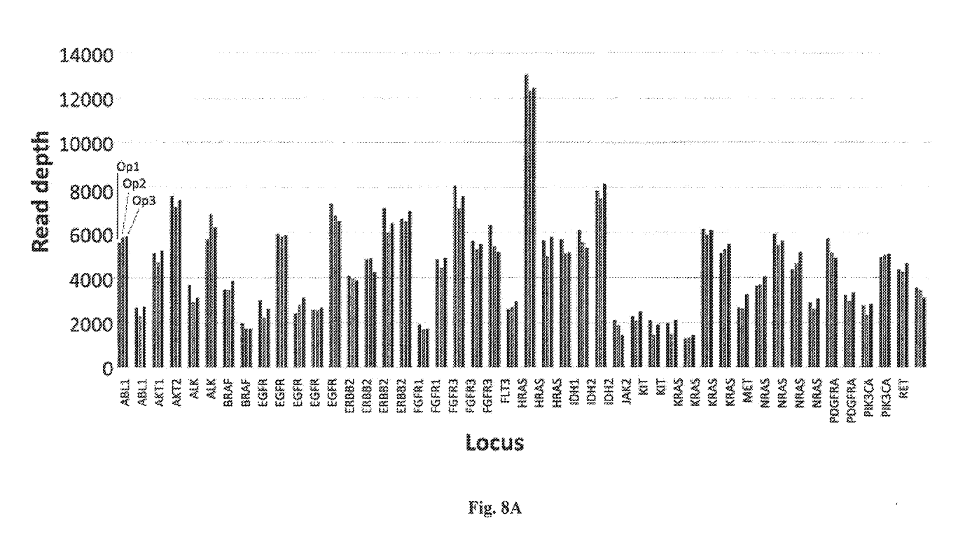

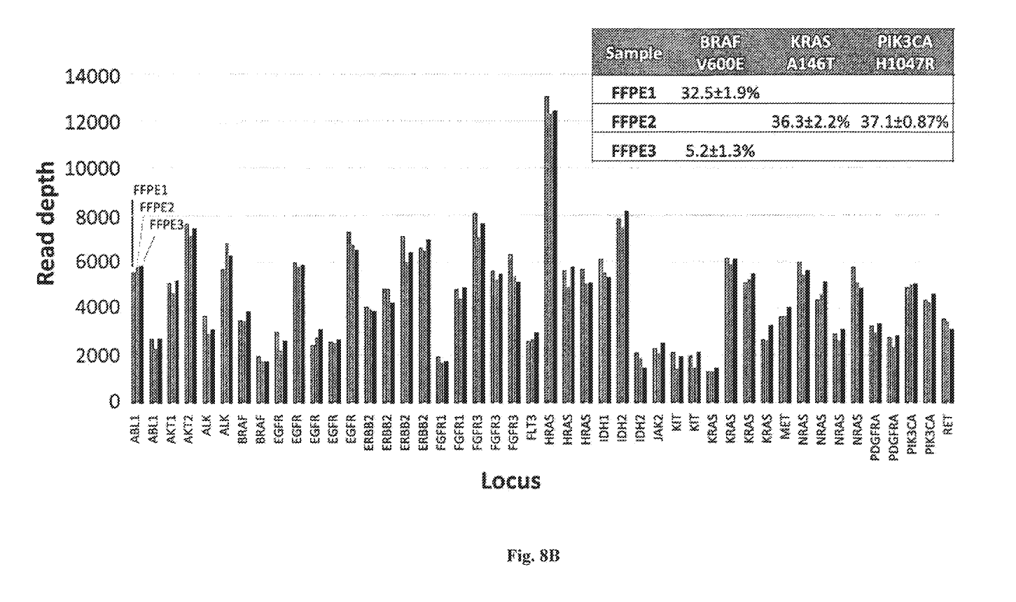

In one aspect, a kit may include a bioinformatics component. In one instance the bioinformatics component is developed with training data sets. In another instance, bioinformatics software will be provided to enable a user to analyze the raw NGS data produced, such as produce by the SuraSeq or QuantideX® Pan Cancer DNA panel. In yet another instance, the software will be a stand-alone tool installed on a user's local machine. In one instance, the software will enable use through a graphical interface presented in the context of a web browser. In another instance, no internet connection will be required to use the software. In yet another instance, a web application will be hosted from a virtual machine that runs in headless mode as a windows service on the machine to which it was installed and will be accessible to any other machine on the local network. In one instance, the software will be HIPAA compliant and/or satisfy the technical safeguards of access control, audit controls, integrity, authentication and transmission security. In another instance, the software will enable a user through a point-click interface to upload raw sequence data from a sequencing instrument, such as a PGM or a MiSeq instrument, upload QuantideX® NGS data and initiate an analysis that produces a concise summary of sample quality control, and/or detected mutations and information to assess the functional consequences of detected variants. In another instance, the software will support export of the results or long term storage. In yet another instance, the bioinformatics analysis is tracked and provided to the user through a project dashboard. In one instance all of the bioinformatics processing takes place on a Linux virtual machine operating a Windows host environment. In another instance, the bioinformatics analysis is trained on and/or provides variability on a specific set of nucleic acid sequences (see FIGS. 8A and B as a non-limiting example). In yet another instance, the variant caller only calls true variants at 400 copy input (see FIG. 9 as a non-limiting example).

### EXAMPLES

The following example is included to demonstrate preferred embodiments of the invention. It should be appreciated by those of skill in the art that the techniques disclosed in the example which follows represent techniques discovered by the inventor to function well in the practice of the invention, and thus can be considered to constitute a preferred mode for its practice. However, those of skill in the art should, in light of the present disclosure, appreciate that many changes can be made in the specific embodiments which are disclosed and still obtain a like or similar result without departing from the spirit and scope of the invention.

### Example 1

### Comparisons of Variant Calling Models with and Without Implementation of Viable Template Count-Specific Features

To assess the impact of viable template count and the viable template count-related features on variant caller performance, we trained a baseline model that included all features except those that were viable template count-specific and a viable template count model that included the baseline features plus the viable template count-specific features (“QuantideX®-enabled caller”). Viable template count was determined using QuantideX® DNA Assay (adapted from Sah et al. 2013). Specifically, the models were trained with the parameters and features noted below. The workflow is demonstrated in FIGS. 3A and B.

### Materials and Methods

DNA Preparation and Sequencing

DNA functionality was assessed by the QuantideX® DNA Assay (adapted from Sah et al., 2013). The QuantideX® DNA Assay guided input into the NGS enrichment step to help ensure the accuracy of variant calling. See FIGS. 3A and B. PCR.based target enrichment was conducted using QuantideX® NGS reagents (modified from Hadd et al., 2013). Sequencing procedures for MiSeq (Illumina) and PCM (ThermoFisher) were followed according to manufacturer's instructions. Mutational status was determined by sequencing with verification by liquid bead array (Luminex) (333) and/or replicate sequencing (467) and considering concordant calls positive after accounting for site and sample-specific background.

Sequencing Analysis

Sequencing analysis was performed by Asuragen's standard preprocessing pipeline, including: amplicon-similarity filtering (based on a banded smith-waterman alignment to the target amplicon set utilizing the Bfast aligner; adapter and PCR-primer trimming; length filtering (remove reads shorter than 20 nucleotides); edge quality trimming (trim low-quality bases (<Q20) from the edge of the amplicon; quality scoring filtering (retain reads with average quality score >20); N-filtering (exclude reads with Ns in them); alignment to GRCh37 using BWA (sw algorithm); GATK indel-realignment and base q-score recalibration using known indels and SNVs from 1000-genomes, dbSNP, and COSMIC (for indel realignments).

Variant calling using VarScan2 (Koboldt et al., 2012) was performed In accordance with recommended protocols (Koboldt et al., 2013).

Model Parameters and Features

The model was trained and performance assessed under 5-fold cross validation. The performance reported is the averaged cross validated scores for positions which were utilized in training, and the model-predicted scores for positions not utilized during training (see below for the set of data used in training). Ada boosted trees as implemented by the “ada” package (version 2.0-3) in R (version 3.0.2) were used with the following parameters:

Iterations: 250

Boosting shrinkage parameter “nu”: 0.05

Sampling fraction for samples taken out-of-bag: 1 (i.e. no random sampling)

Tree depth: 5

Type: real

All other parameters were left as default.

The final bams were scored by two scoring metrics (SuraScore and SuraScoreBB), the data tabulated, and sequence-context metrics added by custom scripts written by Asuragen. This dataset represents over 1280 sequenced samples comprised of the 474 unique samples (some samples were sequenced more than two times).

The set of training data was winnowed by: removing hypotheses where the observed percent variant was <0.5%. (leaving ˜250,000 hypotheses); selecting a random set of 50,000 hypotheses from the 250 k available; taking the union of the random set with all putative somatic variants and 150 randomly-selected putative germline variants for a total of approximately 52,000 hypotheses.

To ensure that the baseline model and the QuantideX®-enabled model were trained on the same dataset, the random number generator seed was manually set to a known seed prior to random selection, providing a consistent random subset of the data.

Training Data Set

A set of 474 unique samples were accumulated including: 8 cancer cell line mixtures, 2 hapmap samples (NA12878 and NA19240), 2 synthetic controls consisting of 46 GBlock (which can be accessed via the world wide web at idt.com/) mutations in the background of genomic DNA at allele frequencies ranging from 1% to 40% mutant, 18 plasma samples, 171 clinical FFPEs, 254 fine needle aspirations (FNAs), and 19 Fresh frozen samples.

These samples were sequenced using one or more of the following targeted-amplicon-sequencing panels: TP53 panel, covering all coding exons for canonical TP53; Suraseq500; Informagen+, a two-pool panel consisting of 68 total amplicons; SuraSeq200; and the QuantideX® Pan Cancer panel, an extension of the Suraseq500 panel in a single-tube format with 46 total amplicons. In total, the sequenced content represents over 6 KB of the human genome, enriched for hotspot regions known to have high clinical relevance in a variety of cancers.

The samples selected were those sequenced at least in duplicate and/or those which were interrogated by some other mutation detection method, including Luminex and digital PCR. Truth was established by comparison to alternative detection methods, where available, and by replicate concordance. In particular, across all replicated sites in replicated samples, a simple model of mean and standard deviation was built in a position-specific fashion based on the lowest 95 percentile of observed percent variants, and candidate mutations called if the observed percent variant was above the mean +2 standard deviations across all replicates. The candidate mutations were further refined by a sample-specific hypothesis criteria wherein the observed mutation must be greater than 2 times the 95th percentile of the observed hypothesis-specific background for the sample in question. The only exception to the above was BRAF V600E, which contained an enriched representation of positives in our set and therefore required a lower position-specific cutoff to call known-positive variants as determined by alternative methodologies.

### Results

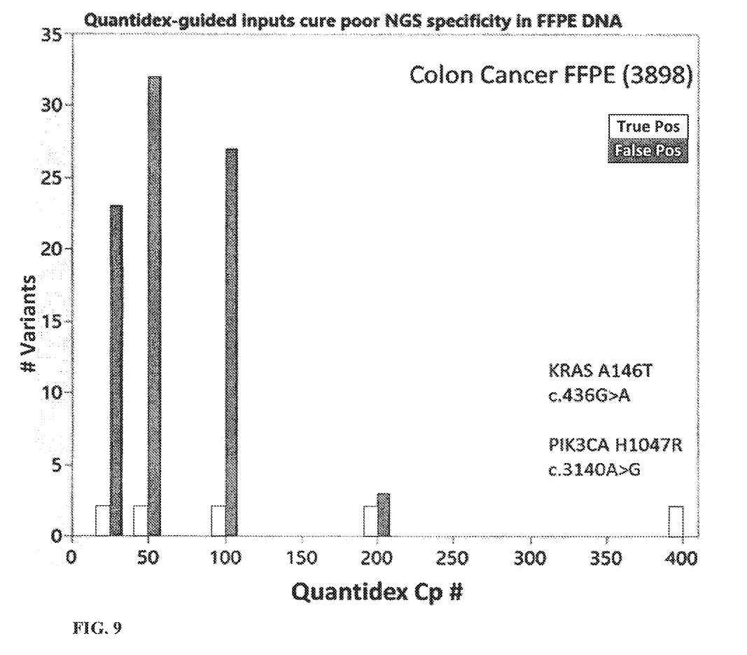

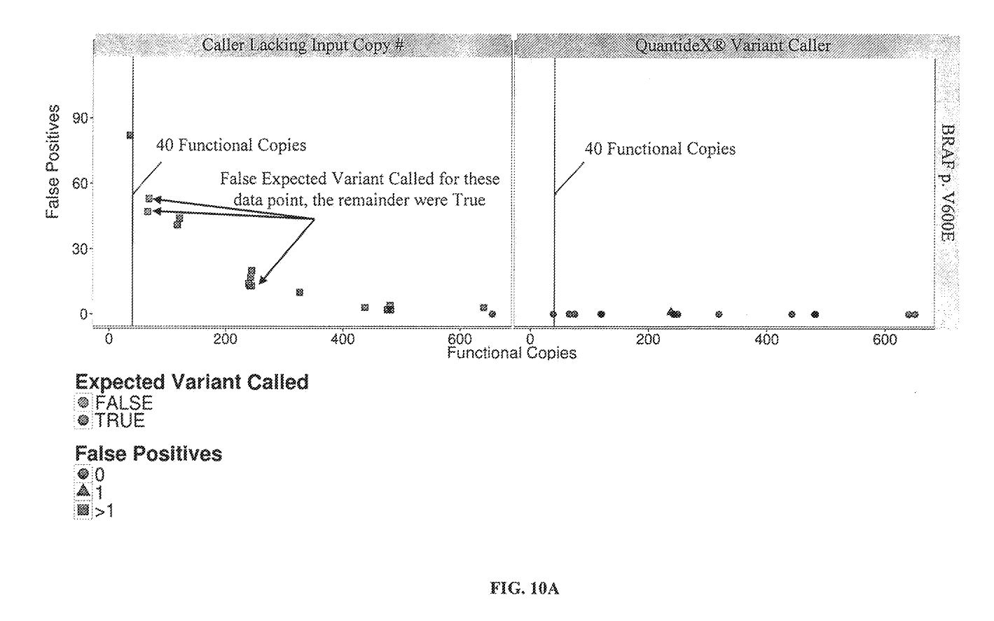

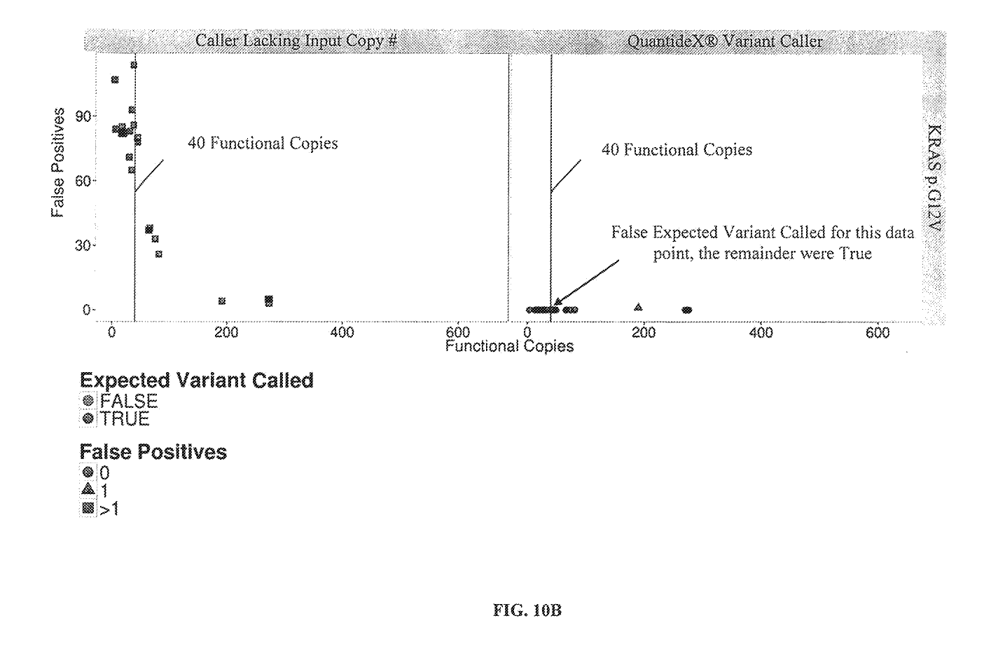

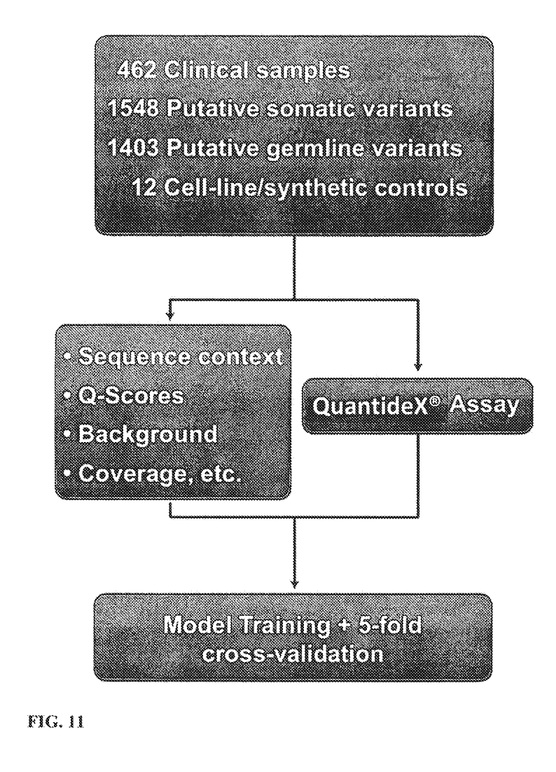

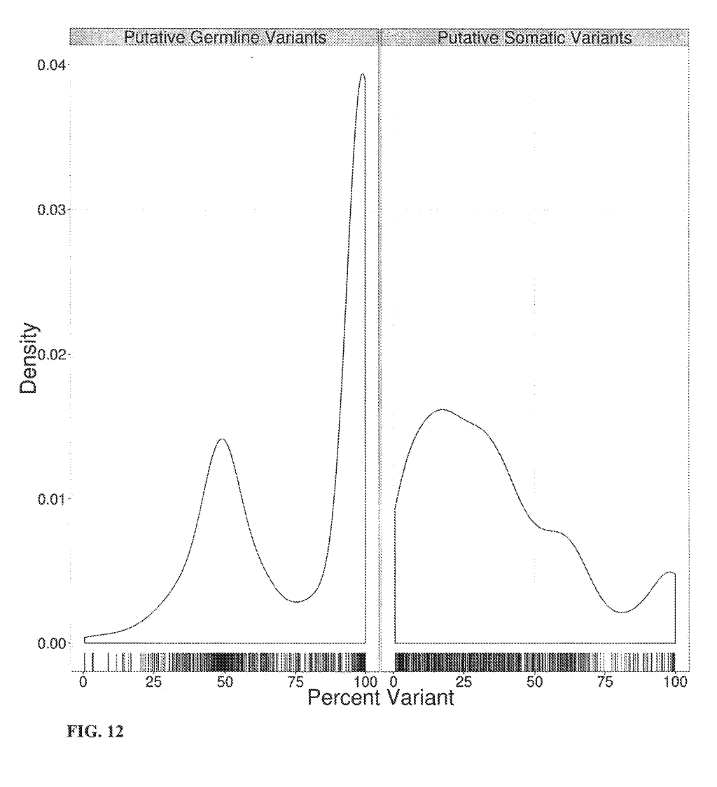

As demonstrated by FIGS. 10A and B, samples with low amplifiable copies put samples at risk for high false positive and high false negative rates. Here samples and designs were used for training a classifier with and without QuantideX® DNA QC Assay data (see FIG. 11 for overview of strategy) that included samples with low viable template count. The variant caller with or without QuantideX® DNA QC Assay data demonstrated positive variant data binned into putative germline variants with a characteristic bimodal allele frequency distribution and putative somatic variants demonstrated a skew to lower abundance variants. See FIG. 12. Taken together, the data suggests a reasonable approximation of somatic vs. germline variants.

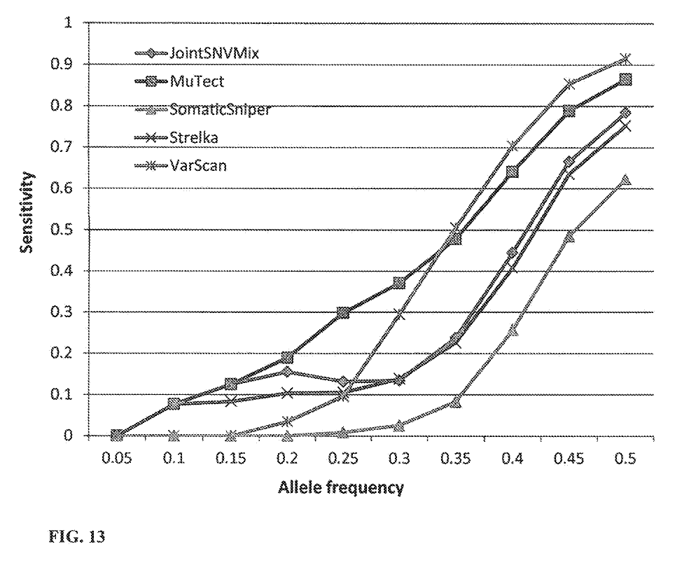

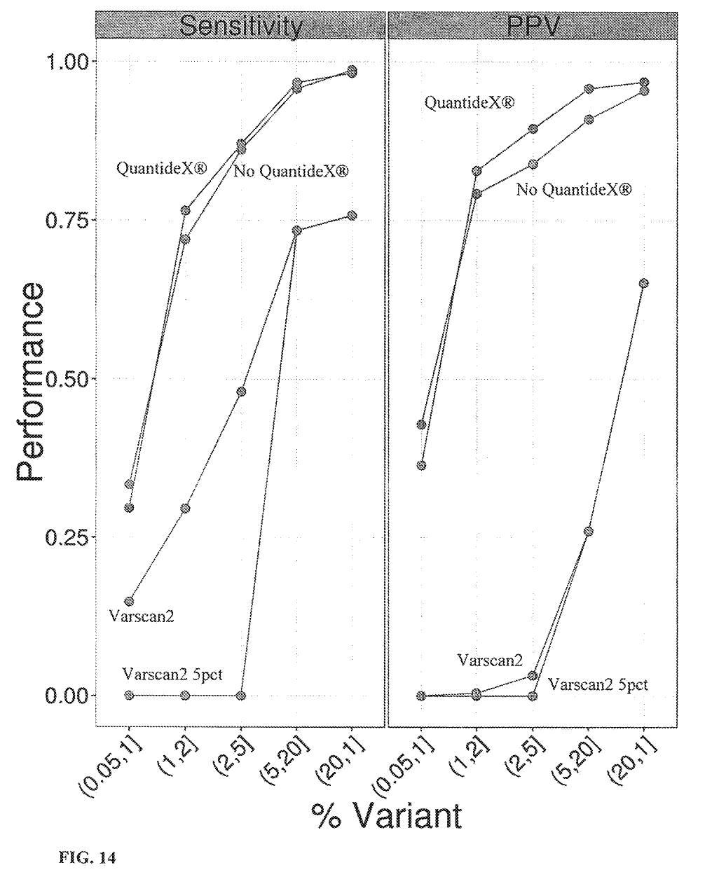

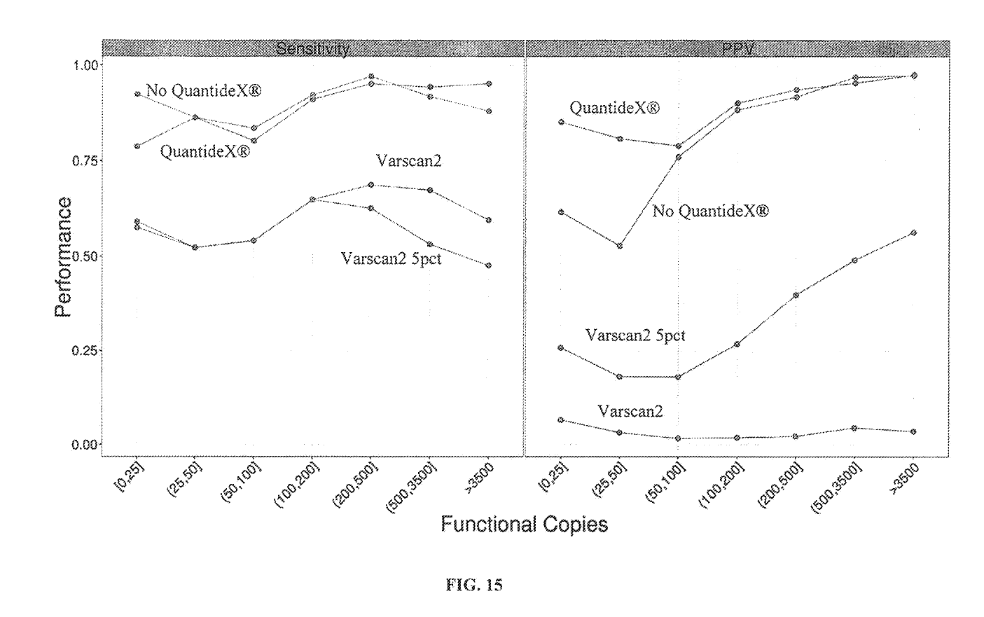

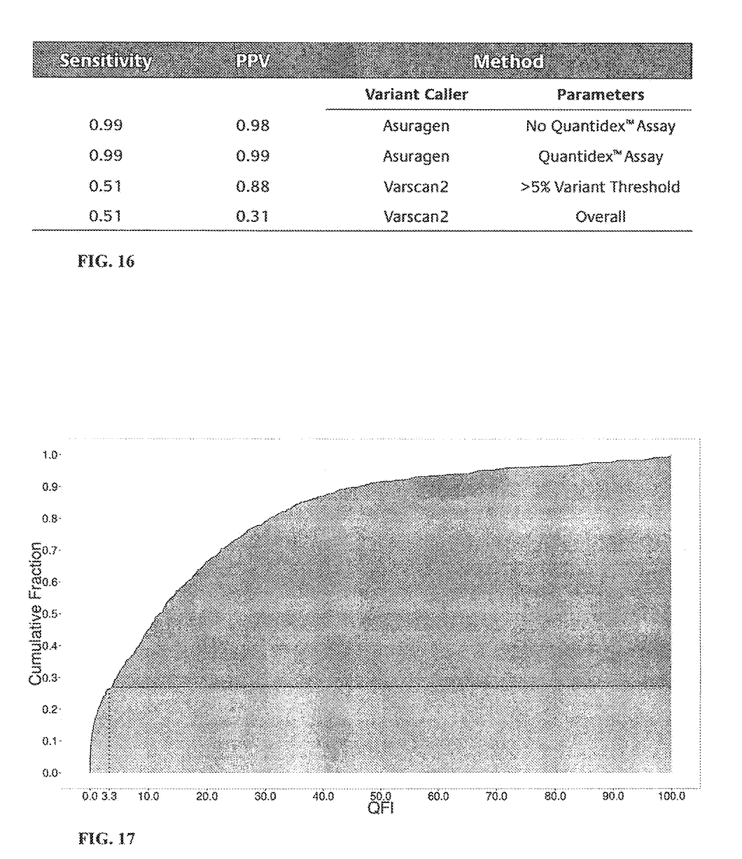

When compared to previously assessed methods, both the baseline model and the QuantideX®-enabled model outperform the competition in sensitivity. FIG. 13 shows sensitivity of other methods assessed independently while FIG. 14 shows sensitivity and PPV for comparable statistics for the method; note that VarScan is the common element between FIG. 13 and FIG. 14 and note that it achieves comparable sensitivity and follows a similar shape in both graphs, note that VarScan significantly gains sensitivity around 20% variant. FIG. 15 demonstrates that that a machine-learning approach with a suitable vector of features can achieve high sensitivity and specificity with respect to allele frequency, better than those achieved by current generation callers, regardless of QuantideX® informatic inclusion. Performance with putative germline variants as demonstrated in FIG. 16 also shows better sensitivity and PPV for both machine-learning approaches.

However, as demonstrated in FIG. 15 when considering sensitivity and performance as a function of copy number, a boost of approximately 50% is observed in the PPV (positive predictive value: the percent of called-variants which are true variants) for samples with <100 functional copies relative to the baseline model. This boost in performance can be directly attributed to the inclusion of the QuantideX® DNA QC Assay copy-number information into the model since all other variables, training schedules, and training parameters were held constant. The 100 copy-number mark is highly relevant because, in a cohort of over 600 FFPE samples assessed, over 27% had fewer than 100 copies per 10-ng of genomic DNA input (10 ng is a common assay input format) (see FIG. 17), illustrating that over 27% of samples would benefit from direct incorporation of QuantideX® QC data into the variant calling model by significantly reducing the number of false positives, even relative to a model in which false positives are already significantly reduced relative to other current-generation variant callers on the market.

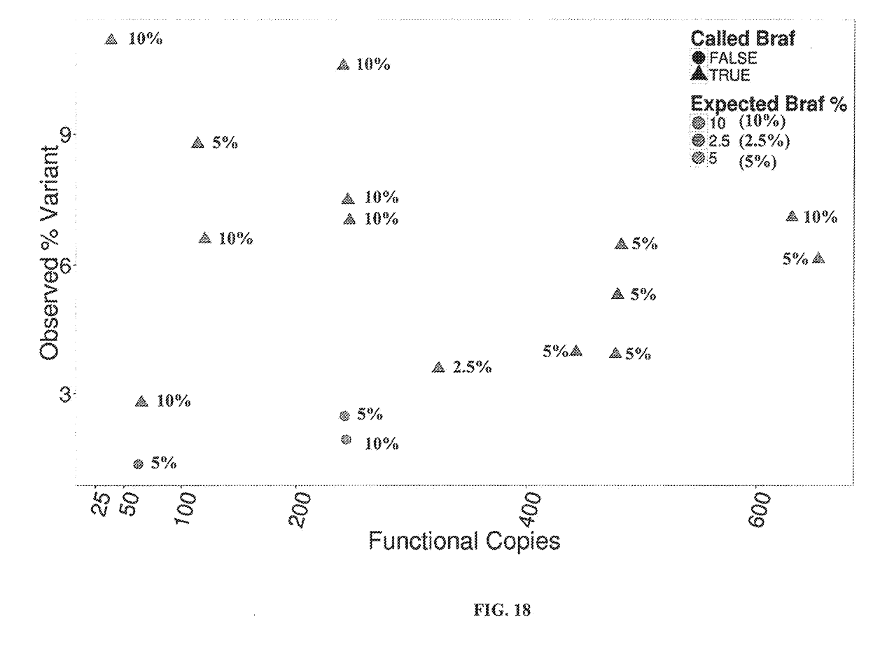

Further, the QuantideX®-enabled caller shows consistent variant detection with low-quantity, low quality residual clinical FFPE DNA. A BRAF V600E-positive FFPE was titrated into the background of a BRAF wild-type FFPE sample to 2.5% variant. Functional copies were titrated between 30 and 660. The samples were called with the trained QuantideX® informatic model. FIGS. 10A and B shows the total number of variant calls. The points are colored by theoretical BRAF percentage and have been jittered to avoid over plotting. FIG. 18 shows observed variant allele frequency vs. functional copy input. The points are shaded by theoretical BRAF percentage and shaped according to BRAF-called (triangles) or not (circles). The QuantideX® caller maintained high sensitivity and PPV, even at low copy inputs and low percent variants. Specifically, the QuantideX® informatic model called BRAF variants in residual clinical FFPE with as few as 34 and 70 functional copies of input, representing just 3.74 (11% variant) and 1.96 (2.8% variant) mutant copies, respectively.

The results reveal that incorporating sample-specific experimental information improves the sensitivity and specificity of mutation detection especially for low-prevalence variants in FFPE and FNA biopsies. The ability to call variants in low-quality and low-quantity DNA samples increases the number of clinical samples that can be processed with high confidence. We also demonstrate variant calling with high sensitivity and PPV for variants present between 0.5% and 10% prevalence for both tumor specimens and defined mixtures of reference cell-line materials. The results underscore the value of a calling system that implements viable template count.

### Example 2

### Asuragen NGS Pan-Cancer DNA Panel

To assess the performance of kits comprising reagents and analysis tools, including a QuantideX®-enabled caller, a NGS pan-cancer DNA panel (FIG. 2B) was developed and tested using cancer-related variants in 21 genes from DNA purified from human tissue or cell-lines. The workflow and specific steps and components are exemplified in FIG. 2A through FIG. 9. The kit supports multiplex next-generation sequencing analysis with an Illumina MiSeq instrument. The kit includes software that analyzes MiSeq data files for the identification of base substitution mutations and small insertions/deletions using a locally integrated bioinformatic pipeline and companion data visualization tools. Specifically, the kit comprises (1) a QuantideX® DNA QC Assay kit comprising primers, probes, ROX, and standards; (2) a QuantideX® Pan Cancer Core Reagents component comprising QuantideX® Pan Cancer primers, a FFPE positive control, a synthetic batch control, Taq, buffer mastermix, diluent; (3) a QuantideX® PurePrep Bead Purification component comprising magnetic beads, elution buffer, and wash buffer; (4) a QuantideX® (MiSeq) component comprising 2× mastermix, ROX, diluent, primers/probes, standards, positive controls, and a calibration means; (5) a QuantideX® Codes MiSeq Index Codes (1-24) primer mix; (6) a QuantideX® Tagging Reagents and Custom MiSeq primers component comprising 2× mastermix, diluent, and custom sequencing primers; and (7) a data pipeline, analysis and reporting tools component comprising an installer, and a web or on-site deployed data analysis package for installation as a local application (FIG. 4). The variant caller is a QuantideX®-enabled caller (QuantideX® Reporter).

Reagents for determine QFI Assay Score and Inhibition Profile using qPCR included 2× Mastermix with reagents combined in a minimum vial set for simple set up and workflow, pre-diluted standards for ease of use and reproducibility, and ROX passive dye for instrument compatibility. A sample cohort mitigation is shown in FIG. 5.

The Asuragen NGS workflow uses 2 steps of PCR: (i) gene-specific amplification with a common sequence concatenated to each primer; (ii) second PCR appending instrument-specific adaptors and index codes are added to the PCR product. Products from individual samples are pooled then clustered onto the flow cell. After imaging, the index codes are used to deconvolute the identity of each amplicon for each sample. The protocol is designed for simple handling and minimum reagents. It includes (1) a primer mix (3545-1) including 92 primer pairs, a 2× PCR Mastermix (3469-1) same as QuantideX®, and sample at fixed volume of 4 mL; and (2) a “Mastermix-free” setup for tagging PCR including oligos as premixture, 2× mastermix (3469-1) and aliquot of gene-specific products.

The kit includes two positive controls, a process control and a FFPE positive control. The process control is formulated from 14 synthetic DNAs admixed with genomic DNA and representing 14 different cancer-related variants. The FFPE positive control is formulated from a BRAF V600E positive and “wild-type” tumor block. Results from our research verification run, MS127, are summarized in Table 1:

TABLE 1

| Operator | Variant | Percent Reads |
|---|---|---|
| 1 | BRAF V600E | 5.3 |
| 2 | BRAF V600E | 3.9 |
| 3 | BRAF V600E | 6.5 |

Library purification used magnetic bead-based purification using the following procedure: bind, wash, elute, designed to reduce <190 bp products and retain specific products. Library quantification is a simple, calibration-curve free qPCR method using competitive PCR with spiked standard for concentration determination. The method works within 100-fold range of the provided standard copy number. The method uses delta Ct to determine the concentration of each library. Other library quantification methods, such as the use of DNA intercalating dyes or qPCR assays that rely on a standard curve to determine the copy number of template molecules in the library, may also be utilized. Instrument loading used Illumina's standard sequencing primers pre-mixed with Asuragen's custom seq primers to specified concentration and supplied with the kit. The kit is designed so that the user pools samples, denatures with PhiX, dilutes and loads to cassette. The user then supplies dual-index code list and links QuantideX® DNA QC results to FASTQ files for analysis.

Bioinformatics used an intuitive bioinformatics software option which enables a user to analyze the raw NGS data produced by the QuantideX® Pan Cancer DNA panel. A prototype user interface was developed to support point-click operation of the pipelines hosted by the virtual machine and visualization of the results reusing SuraSight or QuantideX® reporter GUI components. The prototype allows a user to log in, create an analysis project, upload raw sequence data and initiate an analysis. The status of the analysis is tracked and provided to the user through a project dashboard. Once an analysis completes, a packaged SuraSight or QuantideX® report can be downloaded from the interface. All of this processing takes place on a Linux virtual machine operating in a Windows host environment. A click-through installer has been developed that demonstrates the feasibility of installing the virtual machine on the host through a standard installation wizard.

### Results

A total of 90 total DNA samples were tested using the kit described above. The kit produced a median value of 100% of amplicons within 5× median reads. At a scaled value of 24 samples/run, none of the amplicons in FFPE samples had a coverage depth of <500 reads, NTC ˜4-6 median reads/amplicon. The kit produced 2-6% CV for FFPE mutation quant in multi-operator arm. 5% BRAF FFPE control was detected by all operators (3.9, 5.3, 6.5%). Synthetic controls at 5, 8, 10, and 12% were internally consistent for variant abundance. The kit provided successful detection of DNA samples with known indels and CNV's. There was dose-dependence of library product from inhibited FFPE DNA.

As demonstrated in FIGS. 8A and B, the yield by amplicon, overall coverage and variability between operators highlights performance for the panel. Further, only true variants are called at 400 copy input using QuantideX® informed variant calls, reducing complexity of analysis and confirmation or rejection of false positive results (FIG. 9).

### Example 3

### Asuragen Variant Caller Performance Per Functional Copy

A total of 98 samples were sequenced in a multi-operator, multi-day, multi-run study. Variant caller performance for variants at or above 5% variant allele frequency (VAR) was assessed and split by functional copies input into the library. At 200 copies input, we observed perfect performance, but below 200 copies was associated with increased risk of sensitivity and positive predictive value (PPV). The results are summarized in Table 2:

TABLE 2

| Functional | Number of | | |
|---|---|---|---|
| Copies Input | Expected Variants | Sensitivity | PPV |
| ≤200 | 31 | 0.87 | 0.93 |
| >200 | 340 | 1 | 1 |

### Example 4

### Asuragen Variant Caller Performance on ERBB2 Gene Per Functional Copy

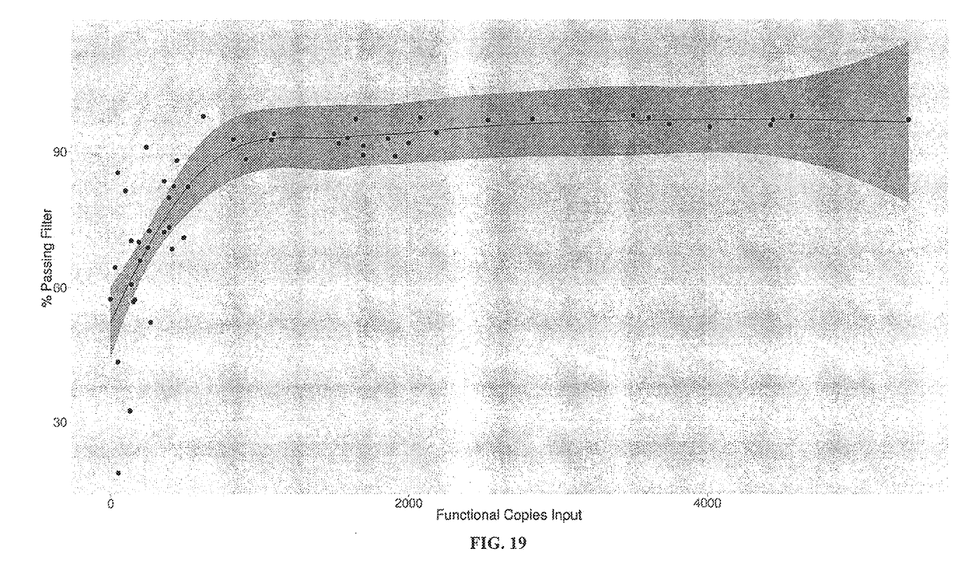

51 paraffin-embedded (FFPE) samples of varying quality were sequenced with a panel targeting the ERBB2 gene. There was a clear relationship between the % of usable sequencing reads (y-axis) and the functional copies input into the sequencing reaction (x-axis), with >1000 copies providing best results, and >200 copies providing adequate results (FIG. 19). Fit line: LOESS smoothed line with 95% CI.

### Example 5

### Asuragen Variant Caller Performance for CNV Compared to ddPCR

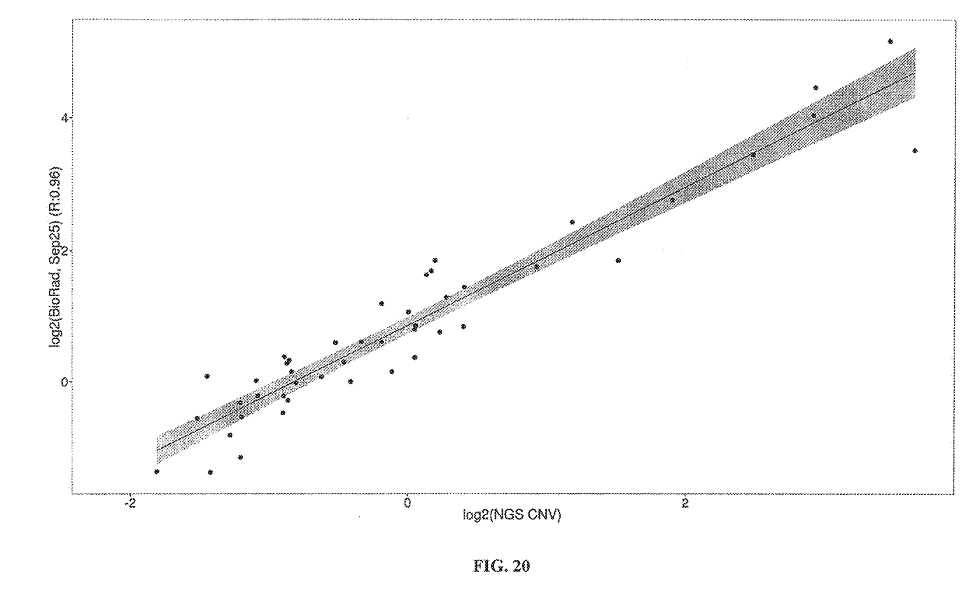

The 51 samples of Example 4, which have known and varied copy number variation (CNV) at the ERBB2 locus, were sequenced using an ERBB2-targeted panel designed with CNV detection capabilities. The same samples were assessed quantitatively for CNVs by droplet digital PCR (ddPCR) (BioRad Sep25) (FIG. 20). The data show strong correlation between the two methods.

### Example 6

### Asuragen Variant Caller Performance for Amplicon Performance Based on Sample Quality

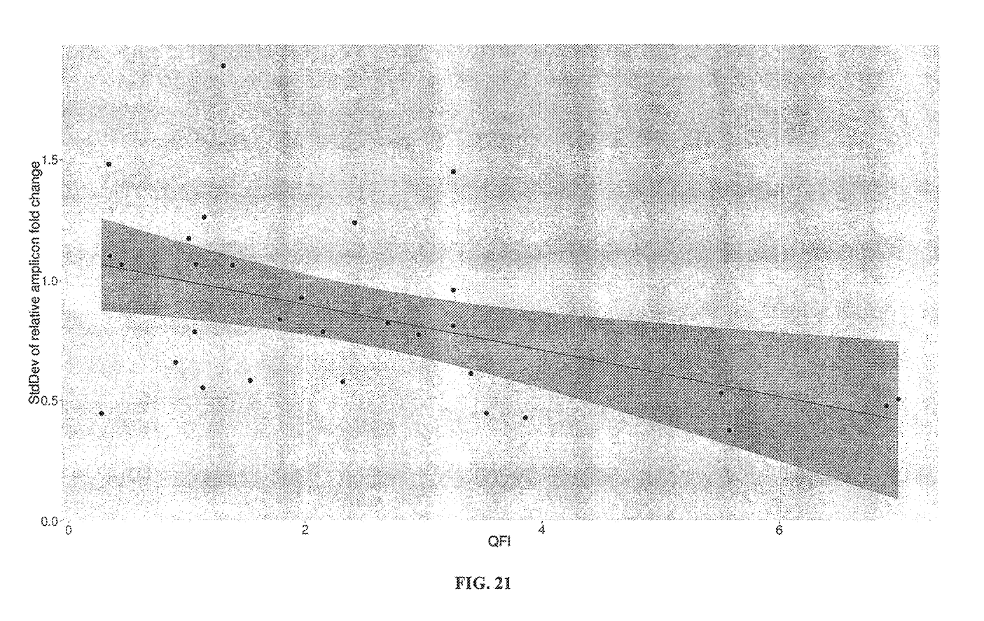

CNV detection in a targeted amplicon panels relies on consistent amplification efficiency of amplicons relative to each other. However, relative amplification efficiency changes as a function of sample quality. Shown is the standard deviation of within-sample relative amplification efficiencies using the 51 samples of Example 4. As the DNA quality score (QFI) decreases, the relative efficiency differences are exacerbated, leading to elevated deviation from expected baselines (FIG. 21). This demonstrates that amplicon performance depends on the sample quality.

### Example 7

### Asuragen Variant Caller Estimated % Functional Copies Comparison to qPCR Based Method

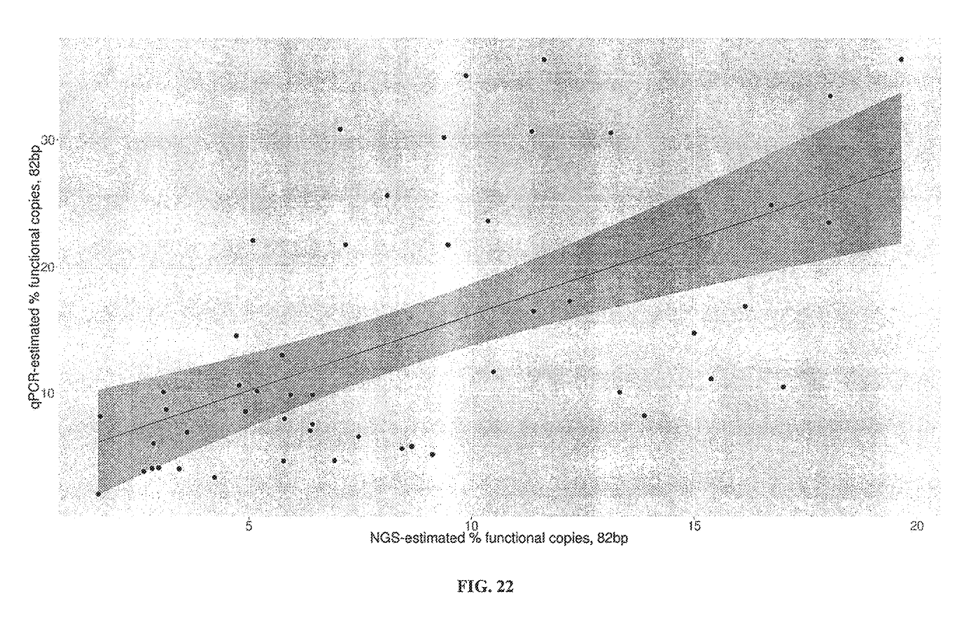

QFI was measured for samples by qPCR for several different amplicon lengths and lesion frequency and % functionality were determined and compared to NGS results of the same samples. The NGS-based approach for estimating sample lesion frequency and, by extension, % functional DNA for any size range (Brisco et al., 2010) compares well with a qPCR-based method for measuring the same information (FIG. 22). This indicates that the pre-sequencing quality control (QC) has a direct impact on relative amplification efficiency and, by extension, the ability to reliably call CNVs.

### Example 8

### Asuragen Variant Caller Compared to Caller Lacking Consideration of Input Copy Number

Low functional copies increase false-positive calls in QC-agnostic caller (FIG. 10 left columns) but not the QuantideX® caller (FIG. 10 right columns) in BRAF (FIG. 10A) and KRAS (FIG. 10B) copy-number titration studies.

### Example 9

### Correlation Between Unique Exonic Content and Four Potential QC Methods

Comparisons of four potential quality control methods for unique exonic content, determined by whole transcriptome RNA-Seq, were performed. The following QC methods were compared: Bioanalyzer (DV200: % of fragments greater than 200 nucleotides), Nanodrop (mass), Qubit RNA (mass) and QuantideX RNA QC (functional copies). R² values for fit to the number of unique exonic reads were assessed for each QC method. The results demonstrate that QuantideX® RNA QC (an RT-qPCR based assay that measures functional RNA copies) provided more accurate results than the other methods. The results are summarized in Table 3.

TABLE 3
CORRELATION BETWEEN UNIQUE EXONIC CONTENT
AND FOUR POTENTIAL QC METHODS

| | | | | QuantideX ® RNA |
|---|---|---|---|---|
| Method | BioAnalyzer | Nanodrop | Qubit | QC |
| Unique Exonic R² | 0.17 | 0.31 | 0.34 | 0.66 |

These results also demonstrate that QuantideX® RNA QC, which uses RNA functional copy assessment, is more predictive of whole transcriptome data quality and of sequencing quality than alternative QC methods.

### Example 10

### Analysis of RNA Functional Copy Assay Can be Used to Rescue Lower Quality Samples and Provide Better Predictions of the Accuracy of Reads

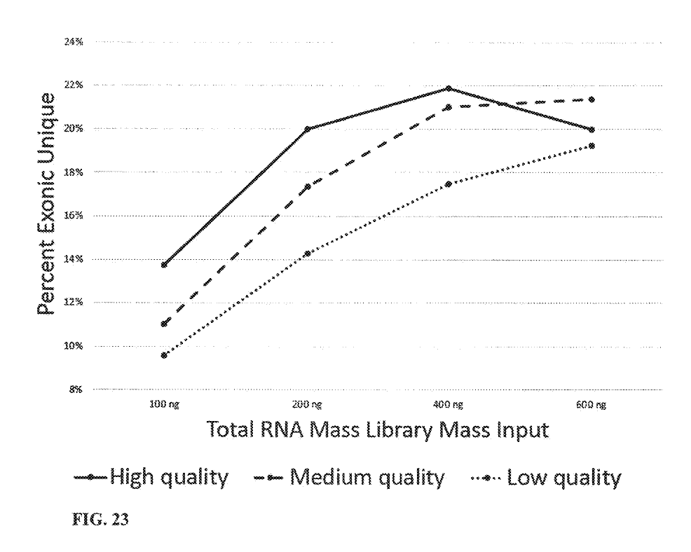

Lower quality FFPE samples (graded by the RNA functional copy assay determined by QuantideX® RNA QC) can be rescued by increasing library mass input (FIG. 23).

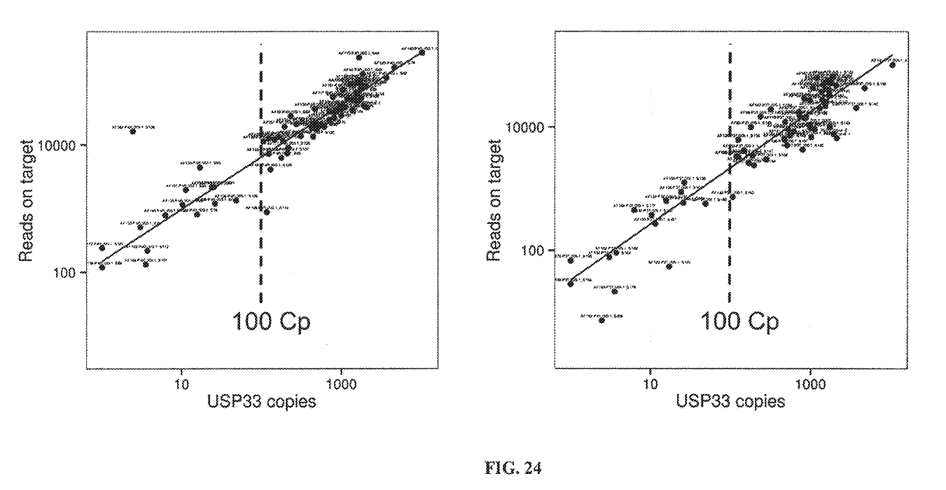

The number of RNA Functional copies also predicts sequencing data quality. Libraries with less than 100 RNA functional copies of endogenous control RNA per 2 ul of RT as determined by QuantideX® RNA QC showed dramatically reduced mapping rates to the intended targets (FIG. 24).

The RNA functional copy number assessment is also predictive of false negative fusion call risks. DNA samples of two fusion genes, RET/PTC1 and PAX8-PPARg, and a negative control (BWH-107A) were used to determine the smallest amount of sample defined by the average functional RNA copies that could be used without receiving a false negative. The results are summarized in Table 4.

TABLE 4

| Average Functional | 8150 | 13418 | BWH-107A |
|---|---|---|---|
| RNA Copies | (RET/PTC1) | (PAX8-PPARg) | (negative) |
| 9398 | RET/PTC1 | PAX8-PPARg | negative |
| 1002 | RET/PTC1 | PAX8-PPARg | negative |
| 569 | RET/PTC1 | PAX8-PPARg | negative |
| 106 | False negative | PAX8-PPARg | negative |
| 50 | False negative | PAX8-PPARg | negative |
| 7 | False negative | False negative | negative |
| 4 | False negative | False negative | negative |

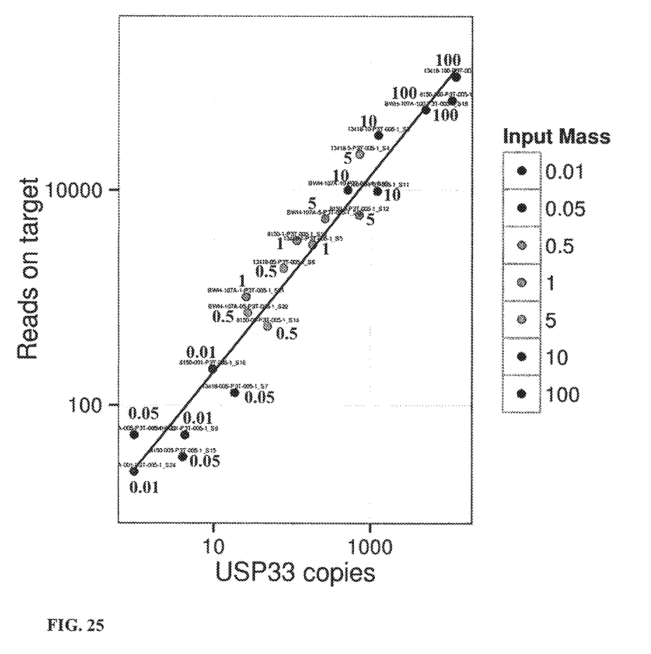

RNA functional copies as determined by QuantideX® RNA QC were plotted according to the reads on target produced by NGS. The plot showed a high correlation between RNA functional copies and the reads on target (FIG. 25). Input mass did not seem to correlate as highly as demonstrated by the spread of similar input masses for the samples tested.

This demonstrates that using RNA functional copy assays before sequencing to modify the amount of sample/number of functional copies per sample can increase the quality of the sequencing data produced. This also demonstrates that considering RNA functional copies in a calling method can better help determine the accuracy of a read. Further, this demonstrates that RNA functional copies is a better predictor of the accuracy of reads than mass of sample used.

All of the apparatuses and/or methods disclosed and claimed herein can be made and executed without undue experimentation in light of the present disclosure. While the apparatuses and methods of this invention have been described in terms of preferred embodiments, it will be apparent to those of skill in the art that variations may be applied to the apparatuses and/or methods and in the steps or in the sequence of steps of the method described herein without departing from the concept, spirit and scope of the invention. Similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pub. No. 2012/0322058
U.S. Pub. No.: 2014/0057793
U.S. Pub. No. 2014/0058681
EP 2602734A1
WO Pub. No. 2013/159145
Akbari M, Hansen M D, Halgunset J, Skorpen F, Krokan H E: Low copy number DNA template can render polymerase chain reaction error prone in a sequence-dependent manner. J Mol Diagn 2005, 7:36-39.
Beltran H, Yelensky R, Frampton G M, Park K, Downing S R, MacDonald T Y, Jarosz M, Lipson D, Tagawa S T, Nanus D M, Stephens P J, Mosquera J M, Cronin M T, Rubin M A: Targeted next-generation sequencing of advanced prostate cancer identifies potential therapeutic targets and disease heterogeneity. Eur Urol 2013, 63:920-926.
Brisco M J, Morely A A: Quantification of RNA integrity and its use for measurement of transcription number. Nucleic Acids Res 2012, 40(18):e144.
Brisco M J, Latham S, Bartley P A, Morley A.: Incorporation of measurement of DNA integrity into qPCR assays. BioTechniques 2010 49:893-897.
Didelot A, Kotsopoulos S K, Lupo A, Pekin D, Li X, Atochin I, Srinivasan P, Zhong Q, Olson J, Link D R, Laurent-Puig P, Blons H, Hutchison J B, Taly V: Multiplex picoliter-droplet digital PCR for quantitative assessment of DNA integrity in clinical samples. Clin Chem 2013, 59:815-823.
Forshew T, Murtaza M, Parkinson C et al.: Noninvasive identification and monitoring of cancer mutations by targeted deep sequencing of plasma DNA. Sci. Transl. Med. 2012, 4(136): 136ra1681.
Gargis A S, Kalman L, Berry M W, Bick D P, Dimmock D P, Hambuch T, Lu F, Lyon E, Voelkerding K V, Zehnbauer B A, et al: Assuring the quality of next-generation sequencing in clinical laboratory practice. Nat Biotechnol 2012, 30:1033-1036.
Hadd A G, Houghton J, Choudhary A, Sah S, Chen L, Marko A C, Sanford T, Buddavarapu K, Krosting J, Garmire L, Wylie D, Shinde R, Beaudenon S, Alexander E K, Mambo E, Adai A T, Latham G J: Targeted, high-depth, next-generation sequencing of cancer genes in formalin-fixed, paraffin-embedded and fine-needle aspiration tumor specimens. J Mol Diagn 2013, 15:234-247.
Koboldt D C, Zhang Q, Larson D E, Shen D, McLellan M D, Lin L, Miller C A, Mardis E R, Ding L, Wilson R: VarScan 2: Somatic mutation and copy number alteration discovery in cancer by exome sequencing. Genome Res 2012, 22(3):568-576.
Menon R, Deng M, Boehm D, Braun M, Fend F, Boehm D, Biskup S, Perner S: Exome Enrichment and SOLiD Sequencing of Formalin Fixed Paraffin Embedded (FFPE) Prostate Cancer Tissue. Int J Mol Sci 2012, 13:8933-8942.
Sah S, Chen L, Houghton J, Kemppainen J, Marko A, Zeigler R, Latham G: Functional DNA quantification guides accurate next-generation sequencing mutation detection in formalin-fixed, paraffin-embedded tumor biopsies. Genome Medicine 2013, 5:77.
Sedlackova T, Repiska G, Celec P, Szemes T, Minarik G: Fragmentation of DNA affects the accuracy of the DNA quantitation by the commonly used methods. Biol Proced Online 2013, 15:5.
Simbolo M, Gottardi M, Corbo V, Fassan M, Mafficini A, Malpeli G, Lawlor R T, Scarpa A: DNA qualification workflow for next generation sequencing of histopathological samples. PLoS One 2013, 8:e62692.
Tuononen K, Mäki-Nevala S, Sarhadi V K, Wirtanen A, Rönty M, Salmenkivi K, Andrews J M, Telaranta-Keerie A I, Hannula S, Lagström S, Ellonen P, Knuuttila A, Knuutila S: Comparison of targeted next-generation sequencing (NGS) and real-time PCR in the detection of EGFR, KRAS, and BRAF mutations on formalin-fixed, paraffin-embedded tumor material of non-small cell lung carcinoma-superiority of NGS. Genes Chromosomes Cancer 2013, 52:503-511.
van Beers E H, Joosse S A, Ligtenberg M J, Fles R, Hogervorst F B, Verhoef S, Nederlof P M: A multiplex PCR predictor for aCGH success of FFPE samples. Br J Cancer 2006, 94:333-337.
Wang F, Wang L, Briggs C, Sicinska E, Gaston S M, Mamon H, Kulke M H, Zamponi R, Loda M, Maher E, Ogino S, Fuchs C S, Li J, Hader C, Makrigiorgos G M: DNA degradation test predicts success in whole-genome amplification from diverse clinical samples. J Mol Diagn 2007, 9:441-451.
Yost S E, Smith E N, Schwab R B et al.: Identification of high-confidence somatic mutations in whole genome sequence of formalin-fixed breast cancer specimens. Nucleic Acids Res 2012, 40(14):e107.

1. A kit for determining a nucleic acid sequence comprising:
(a) a quantitative PCR reagent set capable of being used to determine the viable template count of nucleic acid in a sample;
(b) a multiplexed PCR reagent set capable of being used to amplify multiple target regions in the sample and generating a library of nucleic acid molecules for sequencing;
(c) a tagging PCR reagent set capable of being used to append sequences to the nucleic molecules in the library;
(d) a set of reagents capable of being used to purify and/or normalize the nucleic acid molecules in the library for further amplification prior to sequencing;
(e) a non-transitory machine-readable storage medium comprising instructions that, when executed by a computing device, cause the computing device to identify sequence variants by performing at least the following:
(i) access sequence data associated with the library of nucleic acid molecules; and
(ii) analyze the sequence data to identify sequence variants by taking into account the viable template count associated with the sample.

2. The kit of claim 1, wherein the quantitative PCR reagent set comprises a master mix capable of being used to make a buffer suitable for quantitative PCR.

3. The kit of claim 1 or 2, wherein the quantitative PCR reagent set comprises primers for amplifying a region of nucleic acid in the sample.

4. The kit of any one of claims 1 to 3, wherein the multiplexed PCR reagent set comprises primers configured to amplify at least 5, 10, 15, 20, 25, 30, 35, 40, 45, or 50 genomic regions associated with a disease state or disease propensity.

5. The kit of claim 4, wherein the genomic regions cover at least 50, 100, 200, 300, 400, 500, 600, 700, or 800 loci associated with a disease state or disease propensity.

6. The kit of claim 4 or 5, wherein the disease is cancer.

7. The kit of any one of claims 1 to 6, wherein taking into account a viable template count associated with the sample comprises adjusting the probability of a sequence hypothesis being true based on the value of the viable template count.

8. The kit of any one of claims 1 to 7, wherein taking into account a viable template count associated with the sample comprises downgrading the probability of a sequence hypothesis being true if the variant template count is below a threshold.

9. The kit of any one of claims 1 to 8, wherein taking into account a viable template count associated with the sample comprises upgrading the probability of a sequence hypothesis being true if the variant template count is above a threshold.

10. The kit of any one of claims 1 to 9, wherein taking into account a viable template count associated with the sample comprises adjusting the weight assigned to a feature of a variant calling model based on the value of the viable template count.

11. The kit of any one of claims 1 to 10, wherein taking into account a viable template count associated with the sample comprises adjusting the prior probability of observing a non-reference base as a function of the viable template count.

12. The kit of any one of claims 1 to 11, wherein taking into account a viable template count associated with the sample comprises incorporating the viable template count as a feature of the model.

13. The kit of any one of claims 1 to 12, wherein taking into account a viable template count associated with the sample comprises using a different set of model features to identify sequence variants in the sample if the viable template count lies within a predefined interval.

14. The kit of any one of claims 1 to 13, wherein taking into account a viable template count associated with the sample comprises using an alternative classifier to identify sequence variants if the viable template count lies within a predefined interval.

15. A method of identifying variants in genomic DNA comprising:
(a) performing a quantitative PCR assay to determine the viable template concentration in a sample comprising nucleic acid;
(b) using the viable template concentration to calculate the viable template count in an aliquot of the sample;
(c) performing a PCR reaction to create a library enriched for a nucleic acid segment of interest using the aliquot as a template;
(d) generating sequence data from the library; and
(e) analyzing the sequence data using a computer-based variant calling model that incorporates the viable template count to identify sequence variants in the genomic DNA, wherein incorporating the viable template count comprises configuring the model to do one or more of the following:
adjust the probability of a sequence hypothesis being true based on the value of the viable template count;
downgrade the probability of a sequence hypothesis being true if the variant template count is below a threshold;
upgrade the probability of a sequence hypothesis being true if the variant template count is above a threshold;
adjust the weight assigned to a model feature based on the value of the viable template count;
adjust the prior probability of observing a non-reference base as a function of the viable template count;
incorporate the viable template count as a feature of the model;
identify sequence variants in the sample if the viable template count lies within a predefined interval; and/or
use an alternative classifier to identify sequence variants in the nucleic acid if the viable template count lies within a predefined interval.

16. A method of improving the quality of variant calling of a nucleic acid sample comprising:
(i) determining the amount of functional copies in a sample to be sequenced and
(ii) determining the amount of sample to be used in sequencing based on the amount of functional copies in the sample.

17. The method of claim 16, wherein the functional copies are RNA functional copies.

18. The method of claim 16, wherein the determined amount of sample to be used in sequencing comprises at least 100, 200, 300, 400, or 500 functional copies.

19. A method comprising:
(a) quantifying the viable template count in a sample comprising nucleic acid;
(b) enriching target regions of the nucleic acid to create a library for sequencing;
(c) generating sequence data from the library, wherein the data comprise a plurality of sequence reads;
(d) analyzing the sequence data using a computer-based variant calling model that incorporates the viable template count of the sample in calling a sequence of a target region based on a set of sequence reads.

20. The method of claim 19, wherein the variant calling model is configured to call one or more sequence variations in the sample nucleic acid relative to a reference sequence.

21. The method of claim 20, wherein the one or more sequence variations comprise single nucleotide variants, insertions, deletions, multi-nucleotide substitutions, structural variants, genomic copy number alterations, genomic rearrangements, splicing variants, and/or RNA variants.

22. The method of claim 20 or 21, wherein the one or more sequence variations are associated with a disease state and/or disease propensity.

23. The method of any one of claims 20 to 22, wherein the sequence variations are associated with a pharmacogenomic response such as resistance, sensitivity, and/or toxicity to a drug.

24. The method of any one of claims 19 to 23, wherein the variant calling model is configured to identify quantitative target-specific copy number variations.

25. The method of any of claims claim 19 to 24, wherein the nucleic acid comprises DNA, RNA, and/or total nucleic acid from a biological sample.

26. The method of claim 19 or 25, wherein the nucleic acid comprises genomic DNA.

27. The method of any one of claims 19 to 26, wherein the nucleic acid is derived from one or more of the following: formalin fixed paraffin embedded tissue, tissue collected by fine needle aspiration, frozen tissue, serum, plasma, whole blood, circulating tumor cells, tissue collected by laser capture microdissection, core needle biopsy, cerebrospinal fluid, saliva, buccal swab, stool samples, and urine.

28. The method of any one of claims 19 to 27, wherein the nucleic acid in the sample is heterogeneous.

29. The method of any one of claims 19 to 28, wherein the nucleic acid in the sample is from a mixture of cancer cells and non-cancer cells.

30. The method of any one of claims 19 to 29, wherein the sample has a viable template count below about 10000, 9000, 8000, 7000, 6000, 5000, 4000, 3000, 2000, 1000, 500, 400, 300, 200, 100, or 50.

31. The method of any one of claims 19 to 30, wherein quantifying the viable template count comprises performing a quantitative PCR assay.

32. The method of any one of claims 19 to 31, wherein enriching target regions of the nucleic acid comprises performing a PCR reaction using one or more DNA primer pairs capable of annealing and extending over a target region.

33. The method of claim 32, wherein the PCR reaction is a multiplex reaction.

34. The method of any one of claims 19 to 33, wherein enriching target regions of the nucleic acid comprises performing a capture-hybridization procedure.

35. The method of any one of claims 19 to 34, wherein generating sequence data from the library comprises obtaining a plurality of sequence reads in parallel.

36. The method of any one of claims 19 to 35, wherein the sequence data include multiple sequence reads for each portion of the library.

37. The method of any one of claims 19 to 36, further comprising aligning the sequence data to a reference sequence.

38. The method of any one of claims 19 to 37, wherein the variant calling model is configured to adjust the probability of a sequence hypothesis being true based on the value of the viable template count.

39. The method of claim 38, wherein the variant calling model is configured to downgrade the probability of a sequence hypothesis being true if the variant template count is below a threshold.

40. The method of claim 38, wherein the variant calling model is configured to upgrade the probability of a sequence hypothesis being true if the variant template count is above a threshold.

41. The method of any one of claims 19 to 40, wherein the variant calling model is configured to adjust the weight assigned to a model feature based on the value of the viable template count.

42. The method of any one of claims 38 to 41, wherein the variant calling model is configured to compare the sequence data to a reference sequence.

43. The method of claim 42, wherein the variant calling model is configured to adjust the prior probability of observing a non-reference base as a function of the viable template count.

44. The method of any one of claims 19 to 43, wherein the variant calling model is configured to incorporate the viable template count as a feature of the model.

45. The method of any one of claims 19 to 44, wherein the variant calling model is configured to use a different set of model features to identify sequence variants in the sample if the viable template count lies within a predefined interval.

46. The method of any one of claims 19 to 45, wherein the variant calling model is configured to use an alternative classifier to identify sequence variants in the nucleic acid if the viable template count lies within a predefined interval.

47. The method of any one of claims 19 to 46, wherein the variant calling model is configured to estimate the certainty or probability of error of a variant call as a function of the viable template count for a pre-specified allelic fraction.

48. The method of any one of claims 19 to 47, wherein the variant calling model has an increased positive predictive value (“PPV”), a decreased incidence of false positives, and/or a decreased incidence of false negatives relative to the same variant calling model that does not incorporate the viable template count.

49. The method of any one of claims 19 to 48, wherein the variant calling model has a PPV for samples having a viable template count below 100, 75, 50, or 25 that is at least approximately 5, 10, 15, 20, 25, 30, 35, 40, 45, or 50% higher than the same variant calling model that does not incorporate the viable template count.

50. The method of any one of claims 19 to 49, wherein the variant calling model has a sensitivity for samples having a viable template count below 100 that is no more that 10% less than the same variant calling model that does not incorporate the copy number.

51. The method of any one of claims 19 to 50, wherein the variant calling model has a PPV above 75% for samples having a viable template count below 100, 200, 300, 400, or 500.

52. The method of any one of claims 19 to 51, wherein the variant calling model has a decreased risk of false positives for samples having a viable template count less than 100, 150, or 200.

53. The method of any one of claims 19 to 52, wherein the sample comprises DNA derived from a human subject.

54. The method of claim 53, further comprising determining whether the human subject has a disease or a disease propensity based on the analysis of the sequence data.

55. The method of claim 53 or 54, wherein the disease is cancer.

56. The method of claim any one of claims 53 to 55, further comprising selecting a disease treatment based on the analysis of the sequence data.

57. The method of claim 56, wherein the disease treatment is administering anti-cancer therapy.

58. The method of any one of claims 53 to 57, further comprising electing not to administer a disease treatment based on the analysis of the sequence data.

59. The method of any one of claims 53 to 58, further comprising determining whether a disease treatment would be indicated or contraindicated for the human subject based on the analysis of the sequence data.

60. A method of improving a computer-implemented variant calling model configured to make sequence calls by analyzing sequence data, the method comprising modifying the model by incorporating into the model's analysis of sequence data a viable template count value for an input sample.

61. The method of claim 60, wherein the viable template count value is based on a quantitative PCR assay.

62. The method of claim 61, wherein the quantitative PCR assay measures amplification of a DNA fragment that is of a similar size to PCR amplicons in a library from which sequence data analyzed by the model are derived.

63. The method of claim 60 or 61, wherein incorporating a viable template count into the model's analysis of sequencing data comprises configuring the model to adjust the probability of a sequence hypothesis being true based on the value of the viable template count.

64. The method of any one of claims 60 to 63, wherein incorporating a viable template count into the model's analysis of sequencing data comprises configuring the model to downgrade probability of a sequence hypothesis being true if the variant template count is below a threshold.

65. The method of any one of claims 60 to 64, wherein incorporating a viable template count into the model's analysis of sequencing data comprises configuring the model to upgrade the probability of a sequence hypothesis being true if the variant template count is above a threshold.

66. The method of any one of claims 60 to 65, wherein incorporating a viable template count into the model's analysis of sequencing data comprises configuring the model to adjust the weight assigned to a model feature based on the value of the viable template count.

67. The method of any one of claims 60 to 66, wherein incorporating a viable template count into the model's analysis of sequencing data comprises configuring the model to adjust the prior probability of observing a non-reference base as a function of the viable template count.

68. The method of any one of claims 60 to 67, wherein incorporating a viable template count into the model's analysis of sequencing data comprises configuring the model to incorporate the viable template count as a feature of the model.

69. The method of any one of claims 60 to 68, wherein incorporating a viable template count into the model's analysis of sequencing data comprises configuring the model to use a different set of model features to identify sequence variants in the sample if the viable template count lies within a predefined interval.

70. The method of any one of claims 60 to 69, wherein incorporating a viable template count into the model's analysis of sequencing data comprises configuring the model to use an alternative classifier to identify sequence variants if the viable template count lies within a predefined interval.

71. The method of any one of claims 60 to 70, wherein the modified variant calling model has an increased PPV, a decreased incidence of false positives, and/or a decreased incidence of false negatives relative to the variant calling model before modification.

72. The method of any one of claims 60 to 71, wherein the modified variant calling model has a PPV for input DNA with a copy number below 100, 75, 50, or 25 that is at least approximately 5, 10, 15, 20, 25, 30, 35, 40, 45, or 50% higher than the variant calling model before modification.

73. The method of claim 72, wherein the modified variant calling model has a sensitivity for input samples having a viable template count less than 100 that is no more that 10% less than the sensitivity of the variant calling model before modification.

74. The method of any one of claims 60 to 73, wherein the modified variant calling model has a PPV above 75% for input aliquots having a viable template count below 100, 200, 300, 400, or 500.

75. The method of any one of claims 60 to 74, wherein the modified variant calling model has a decreased risk of false positives for input aliquots having a viable template count less than 100, 150, or 200 relative to the model before modification.

76. The method of any one of claims 60 to 75, further comprising training the model using a panel of known variants and sequencing data derived from input samples with varying viable template count values, including samples with fewer than about 100 functional DNA copies and samples with more than about 500 functional DNA copies.

77. A non-transitory machine-readable storage medium comprising instructions that, when executed by a computing device, cause the computing device to perform at least the following:
(a) access sequence data associated with a library of nucleic acid molecules, wherein the library is generated from a nucleic acid input sample; and
(b) analyze the sequence data to identify sequence variants by taking into account a viable template count associated with the input sample.

78. The storage medium of claim 77, wherein the library comprises nucleic acid molecules enriched from the nucleic acid input sample by PCR and/or capture hybridization.

79. The storage medium of claim 78, wherein the enriched nucleic acid molecules are associated with a disease state, a disease propensity, and/or a pharmacogenomic response to drug treatment.

80. The storage medium of any one of claims 77 to 79, wherein the viable template count has been calculated by a quantitative PCR assay.

81. The storage medium of any one of claims 77 to 80, wherein the nucleic acid input sample is derived from a biological sample selected from one or more of the following: formalin fixed paraffin embedded tissue, tissue collected by fine needle aspiration, frozen tissue, serum, plasma, whole blood, circulating tumor cells, tissue collected by laser capture microdissection, core needle biopsy, cerebrospinal fluid, saliva, buccal swab, stool samples, and urine.

82. The storage medium of any one of claims 77 to 81, wherein the input nucleic acid comprises DNA, RNA, and/or total nucleic acid from a biological sample.

83. The storage medium of any one of claims 77 to 82, wherein the input nucleic acid comprises genomic DNA.

84. The storage medium of any one of claims 77 to 83, wherein taking into account a viable template count associated with the input sample comprises adjusting the probability of a sequence hypothesis being true based on the value of the viable template count.

85. The storage medium of any one of claims 77 to 84, wherein taking into account a viable template count associated with the input sample comprises downgrading the probability of a sequence hypothesis being true if the variant template count is below a threshold.

86. The storage medium of any one of claims 77 to 85, wherein taking into account a viable template count associated with the input sample comprises upgrading the probability of a sequence hypothesis being true if the variant template count is above a threshold.

87. The storage medium of any one of claims 77 to 86, wherein taking into account a viable template count associated with the input sample comprises adjusting the weight assigned to a feature of a variant calling model based on the value of the viable template count.

88. The storage medium of any one of claims 77 to 87, wherein taking into account a viable template count associated with the input sample comprises adjusting the prior probability of observing a non-reference base as a function of the viable template count.

89. The storage medium of any one of claims 77 to 88, wherein taking into account a viable template count associated with the input sample comprises incorporating the viable template count as a feature of the model.

90. The storage medium of any one of claims 77 to 89, wherein taking into account a viable template count associated with the input sample comprises using a different set of model features to identify sequence variants in the sample if the viable template count lies within a predefined interval.

91. The storage medium of any one of claims 77 to 90, wherein taking into account a viable template count associated with the input sample comprises using an alternative classifier to identify sequence variants if the viable template count lies within a predefined interval.


SEQUENCE LISTING (8 sequences)

SEQ ID NO: 1 - DNA, 71 bp, Artificial Sequence
  Synthetic Primer
aatgatacggcgaccaccgagatctacacctagtcatacacgagactggacactgacgacatggttctaca

SEQ ID NO: 2 - DNA, 71 bp, Artificial Sequence
  Synthetic Primer
aatgatacggcgaccaccgagatctacacagtgacacacacgagactggacactgacgacatggttctaca

SEQ ID NO: 3 - DNA, 68 bp, Artificial Sequence
  Synthetic Primer
caagcagaagacggcatacgagatctacagagacgttcaggaggtgtacggtagcagagacttggtct

SEQ ID NO: 4 - DNA, 68 bp, Artificial Sequence
  Synthetic Primer
caagcagaagacggcatacgagatcagatcgtctgttcaggaggtgtacggtagcagagacttggtct

SEQ ID NO: 5 - DNA, 10 bp, Artificial Sequence
  Synthetic Primer
gtacagagac

SEQ ID NO: 6 - DNA, 10 bp, Artificial Sequence
  Synthetic Primer
cagatcgtct

SEQ ID NO: 7 - DNA, 10 bp, Artificial Sequence
  Synthetic Primer
gtctctgtac

SEQ ID NO: 8 - DNA, 10 bp, Artificial Sequence
  Synthetic Primer
agacgatctg